(19) United States
(12) Patent Application Publication
(10) Pub. No.: US 2024/0260465 A1
(43) Pub. Date: Aug. 1, 2024

(54) PHOTOELECTRIC CONVERSION ELEMENT, IMAGING ELEMENT, OPTICAL SENSOR, AND COMPOUND

(71) Applicant: FUJIFILM Corporation, Tokyo (JP)

(72) Inventors: Kazuhei KANEKO, Ashigarakami-gun (JP); Hiroki SUGIURA, Ashigarakami-gun (JP); Takehiro YAMANE, Ashigarakami-gun (JP)

(73) Assignee: FUJIFILM Corporation, Tokyo (JP)

(21) Appl. No.: 18/616,826

(22) Filed: Mar. 26, 2024

Related U.S. Application Data
(63) Continuation of application No. PCT/JP2022/035904, filed on Sep. 27, 2022.

(30) Foreign Application Priority Data
Sep. 29, 2021 (JP) 2021-159628

(51) Int. Cl. H10K 85/60; C07D 209/96; C07D 401/06; C07D 403/06; C07D 409/06; H10K 30/30; H10K 85/20

(52) U.S. Cl. CPC H10K 85/6572; C07D 209/96; C07D 401/06; C07D 403/06; C07D 409/06; H10K 85/211; H10K 85/615; H10K 85/654; H10K 85/6576; H10K 30/30

(57) ABSTRACT

An object of the present invention is to provide a photoelectric conversion element in which variation in dark current is suppressed and which has excellent heat resistance. Another object of the present invention is to provide an imaging element, an optical sensor, and a compound.

The photoelectric conversion element includes a conductive film, a photoelectric conversion film, and a transparent conductive film in this order, in which the photoelectric conversion film contains a compound represented by Formula (1).

### CROSS-REFERENCE TO RELATED APPLICATIONS

This application is a Continuation of PCT International Application No. PCT/JP2022/035904 filed on Sep. 27, 2022, which claims priority under 35 U.S.C. § 119(a) to Japanese Patent Application No. 2021-159628 filed on Sep. 29, 2021. The above applications are hereby expressly incorporated by reference, in their entirety, into the present application.

### 1. FIELD OF THE INVENTION

The present invention relates to a photoelectric conversion element, an imaging element, an optical sensor, and a compound.

### 2. DESCRIPTION OF THE RELATED ART

In recent years, the development of an element (for example, an imaging element) having a photoelectric conversion film has been progressing.

For example, it is disclosed in WO2019/189134A that an indolenine compound is used as a material applied to a photoelectric conversion element. Specifically, for example, compounds having the following structures are disclosed.

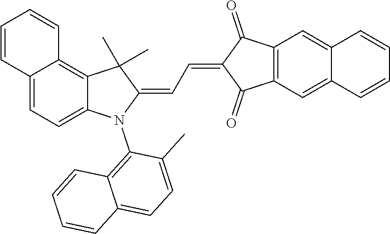 [SMILES: Cc1ccc2ccccc2c1N1/C(=C/C=C2C(=O)c3cc4ccccc4cc3C2=O)C(C)(C)c2c1ccc1ccccc21]

### SUMMARY OF THE INVENTION

In recent years, along with the demand for improving the performance of imaging elements, optical sensors, and the like, further improvements are required for various characteristics required for photoelectric conversion elements used therein.

The present inventors have produced a plurality of photoelectric conversion elements using the compound disclosed in WO2019/189134A, have examined the photoelectric conversion elements, and have found that there is a case where the value of the dark current greatly varies for each photoelectric conversion element (in other words, the value of the dark current has variation). That is, it was clarified that there is room for examination of a photoelectric conversion element that can be stably manufactured by suppressing variation in dark current.

In addition, the photoelectric conversion element is also required to have a property that the photoelectric conversion efficiency does not easily fluctuate even in a case of being exposed to a high temperature (in other words, the photoelectric conversion element has excellent heat resistance) as basic performance.

Therefore, an object of the present invention is to provide a photoelectric conversion element in which variation in dark current is suppressed and which has excellent heat resistance. Another object of the present invention is to provide an imaging element, an optical sensor, and a compound.

The inventors of the present invention have conducted extensive studies on the above-described problems. As a result, the inventors have found that it is possible to solve the above-described problems by applying the compound having a predetermined structure to the photoelectric conversion film, and have completed the present invention.
[1] A photoelectric conversion element comprising, in the following order, a conductive film, a photoelectric conversion film, and a transparent conductive film, the photoelectric conversion film containing a compound represented by Formula (1).
[2] The photoelectric conversion element according to [1], in which the substituent in B¹ includes no substituent having a Hammett's substituent constant 6p of 0.05 or less.
[3] The photoelectric conversion element according to [1] or [2], in which A¹ represents the group represented by Formula (A-1).
[4] The photoelectric conversion element according to any one of [1] to [3], in which A¹ represents a group represented by Formula (A-3).
[5] The photoelectric conversion element according to any one of [1] to [4], in which Z¹ represents an oxygen atom.
[6] The photoelectric conversion element according to any one of [1] to [5], in which the photoelectric conversion film further contains an n-type organic semiconductor, and
the photoelectric conversion film has a bulk hetero structure formed in a state where the compound represented by Formula (1) and the n-type organic semiconductor are mixed.
[7] The photoelectric conversion element according to [6], in which the n-type organic semiconductor contains fullerenes selected from the group consisting of a fullerene and a derivative of the fullerene.
[8] The photoelectric conversion element according to any one of [1] to [7], in which the photoelectric conversion film further includes a p-type organic semiconductor.
[9] The photoelectric conversion element according to any one of [1] to [8], in which the photoelectric conversion film further contains a coloring agent.
[10] The photoelectric conversion element according to any one of [1] to [9], further comprising one or more interlayers between the conductive film and the transparent conductive film, in addition to the photoelectric conversion film.
[11] An imaging element comprising the photoelectric conversion element according to any one of [1] to [10].
[12] An optical sensor comprising the photoelectric conversion element according to any one of [1] to [10].
[13] A compound represented by Formula (1).
[14] The compound according to [13], in which the substituent in B¹ includes no substituent having a Hammett's substituent constant 6p of 0.05 or less.
[15] The compound according to [13] or [14], in which A¹ represents the group represented by Formula (A-1).
[16] The compound according to any one of [13] to [15], in which A¹ represents a group represented by Formula (A-3).
[17] The compound according to any one of [13] to [16], in which Z¹ represents an oxygen atom.

According to the present invention, it is possible to provide a photoelectric conversion element in which variation in dark current is suppressed and which has excellent heat resistance.

In addition, according to the present invention, it is possible to provide the imaging element, the optical sensor, and the compound.

### BRIEF DESCRIPTION OF THE DRAWINGS

FIG. 1 is a schematic cross-sectional view illustrating a configuration example of a photoelectric conversion element.

FIG. 2 is a schematic cross-sectional view illustrating a configuration example of the photoelectric conversion element.

### DESCRIPTION OF THE PREFERRED EMBODIMENTS

Hereinafter, suitable embodiments of a photoelectric conversion element of the present invention will be described.

In the present specification, the numerical range represented by “to” means a range including numerical values denoted before and after “to” as a lower limit value and an upper limit value.

In the present specification, in a case where there are plural substituents, linking groups, and the like (hereinafter, referred to as “substituents and the like”) represented by specific symbols, or a case where a plurality of substituents and the like are specified all together, each of the substituents and the like may be the same or may be different from each other. This also applies to a case of specifying the number of substituents and the like.

In the present specification, a hydrogen atom may be a light hydrogen atom (an ordinary hydrogen atom) or a deuterium atom (for example, a double hydrogen atom and the like).

In the present specification, a “substituent” includes a group exemplified by a substituent W described later, unless otherwise specified.

### (Substituent W)

A substituent W in the present specification will be described below.

Examples of the substituent W include a halogen atom (for example, a fluorine atom, a chlorine atom, a bromine atom, an iodine atom, and the like), an alkyl group (including a cycloalkyl group, a bicycloalkyl group, and a tricycloalkyl group), an alkenyl group (including a cycloalkenyl group and a bicycloalkenyl group), an alkynyl group, an aryl group, a heteroaryl group (may also be referred to as a heterocyclic group), a cyano group, a nitro group, an alkoxy group, an aryloxy group, a silyloxy group, a heterocyclic oxy group, an acyloxy group, a carbamoyloxy group, an alkoxycarbonyloxy group, an aryloxycarbonyloxy group, a secondary or tertiary amino group (including an anilino group), an alkylthio group, an arylthio group, a heterocyclic thio group, an alkyl or an arylsulfinyl group, an alkyl or an arylsulfonyl group, an acyl group, an aryloxycarbonyl group, an alkoxycarbonyl group, an aryl or a heterocyclic azo group, an imide group, a phosphino group, a phosphinyl group, a phosphinyloxy group, a phosphinylamino group, a phosphono group, a silyl group, a carboxy group, a phosphoric acid group, a sulfonic acid group, a hydroxy group, a thiol group, an acylamino group, a carbamoyl group, a ureido group, a boronic acid group, and a primary amino group. Each of the above-described groups may further have a substituent (for example, one or more groups of each of the above-described groups), as possible. For example, an alkyl group which may have a substituent is also included as a form of the substituent W.

In addition, in a case where the substituent W has a carbon atom, the number of carbon atoms of the substituent W is, for example, 1 to 20.

The number of atoms other than a hydrogen atom included in the substituent W is, for example, 1 to 30.

In addition, the specific compound described later preferably does not contain, as a substituent, a carboxy group, a salt of a carboxy group, a salt of a phosphoric acid group, a sulfonic acid group, a salt of a sulfonic acid group, a hydroxy group, a thiol group, an acylamino group, a carbamoyl group, a ureido group, or a boronic acid group (—B(OH)₂) and/or a primary amino group.

In the present specification, examples of the halogen atom include a fluorine atom, a chlorine atom, a bromine atom, and an iodine atom.

In the present specification, unless otherwise specified, the number of carbon atoms of the alkyl group is preferably 1 to 20, more preferably 1 to 10, and still more preferably 1 to 6.

The alkyl group may be any of linear, branched, or cyclic.

Examples of the alkyl group include a methyl group, an ethyl group, a n-propyl group, an i-propyl group, a n-butyl group, a t-butyl group, a n-hexyl group, a cyclopentyl group, and the like.

In addition, the alkyl group may be, for example, a cycloalkyl group, a bicycloalkyl group, or a tricycloalkyl group, and may have a cyclic structure thereof as a partial structure.

In the alkyl group which may have a substituent, a substituent which may be contained in the alkyl group is not particularly limited, an example thereof includes the substituent W, and an aryl group (preferably having 6 to 18 carbon atoms, and more preferably having 6 carbon atoms), a heteroaryl group (preferably having 5 to 18 carbon atoms, and more preferably having 5 and 6 carbon atoms), or a halogen atom (preferably a fluorine atom or a chlorine atom) is preferable.

In the present specification, the alkyl group may be shown to be classified into two groups of a linear or branched alkyl group and a cycloalkyl group. Even in such a case, the suitable aspects of the above-mentioned alkyl group can be cited. As the linear or branched alkyl group, unless otherwise specified, the aspect of the linear or branched alkyl group in the above description of the alkyl group is preferable, and as the cycloalkyl group, the aspect of the cyclic alkyl group in the above description of the alkyl group is preferable.

In the present specification, unless otherwise specified, the above-described alkyl group is preferable as an alkyl group moiety in the alkoxy group. The alkyl group moiety in the alkylthio group is preferably the above-described alkyl group.

In the alkoxy group which may have a substituent, the substituent which may be contained in the alkoxy group includes the same examples as the substituent in the alkyl group which may have a substituent. In the alkylthio group which may have a substituent, the substituent which may be contained in the alkylthio group includes the same examples as the substituent in the alkyl group which may have a substituent.

In the present specification, the alkenyl group may be any of linear, branched, or cyclic, unless otherwise specified. The alkenyl group preferably has 2 to 20 carbon atoms. In the alkenyl group which may have a substituent, the substituent which may be contained in the alkenyl group includes the same examples as the substituent in the alkyl group which may have a substituent.

In the present specification, an alkynyl group may be any of linear, branched, or cyclic, unless otherwise specified. The alkynyl group preferably has 2 to 20 carbon atoms. In the alkynyl group which may have a substituent, the substituent which may be contained in the alkynyl group includes the same examples as the substituent in the alkyl group which may have a substituent.

In the present specification, unless otherwise specified, examples of a silyl group which may have a substituent include a group represented by —Si(R^{S1})(R^{S2})(R^{S3}). R^{S1}, R^{S2}, and R^{S3} each independently represent a hydrogen atom or a substituent, and preferably represent an alkyl group which may have a substituent, an alkoxy group which may have a substituent, an alkylthio group which may have a substituent, an aryl group which may have a substituent, or a heteroaryl group which may have a substituent.

In the present specification, an aromatic ring may be any of a monocyclic ring or a polycyclic ring (for example, 2 to 6 rings or the like), unless otherwise specified. The monocyclic aromatic ring is an aromatic ring having only one aromatic ring structure as a ring structure. The polycyclic (for example, 2 to 6 rings or the like) aromatic ring is an aromatic ring formed by a plurality of (for example, 2 to 6 or the like) aromatic ring structures being fused, as a ring structure.

The number of ring member atoms of the aromatic ring is preferably 5 to 15.

The aromatic ring may be an aromatic hydrocarbon ring or an aromatic heterocyclic ring.

In a case where the aromatic ring is an aromatic heterocyclic ring, the number of heteroatoms contained as ring member atoms is, for example, 1 to 10. Examples of the heteroatoms include a nitrogen atom, a sulfur atom, an oxygen atom, a selenium atom, a tellurium atom, a phosphorus atom, a silicon atom, and a boron atom.

Examples of the aromatic hydrocarbon ring include a benzene ring, a naphthalene ring, an anthracene ring, and a phenanthrene ring.

Examples of the aromatic heterocyclic ring include a pyridine ring, a pyrimidine ring, a pyridazine ring, a pyrazine ring, a triazine ring (1,2,3-triazine ring, 1,2,4-triazine ring, 1,3,5-triazine ring, or the like), and a tetrazine ring (1,2,4,5-tetrazine ring or the like), a quinoxaline ring, a pyrrole ring, a furan ring, a thiophene ring, an imidazole ring, an oxazole ring, a thiazole ring, a benzopyrrole ring, a benzofuran ring, a benzothiophene ring, a benzoimidazole ring, a benzoxazole ring, a benzothiazole ring, a naphthopyrrole ring, a naphthofuran ring, a naphthothiophene ring, a naphthimidazole ring, a naphthoxazole ring, a 3H-pyrrolidine ring, a pyrroloimidazole ring (a 5H-pyrrolo[1,2-a]imidazole ring and the like), an imidazooxazole ring (an imidazo[2,1-b]oxazole ring and the like), a thienothiazole ring (a thieno[2,3-d]thiazole ring and the like), a benzothiadiazole ring, a benzodithiophene ring (benzo[1,2-b:4,5-b′]dithiophene ring or the like), a thienothiophene ring (thieno[3,2-b]thiophene ring or the like), a thiazolothiazole ring (thiazolo[5,4-d]thiazole ring or the like), a naphthodithiophene ring (naphtho[2,3-b:6,7-b′]dithiophene ring, a naphtho[2,1-b:6,5-b′]dithiophene ring, a naphtho[1,2-b:5,6-b′]dithiophene ring, a 1,8-dithiadicyclopenta[b,g]naphthalene ring, or the like), a benzothienobenzothiophene ring, a dithieno[3,2-b:2′,3′-d]thiophene ring, a 3,4,7,8-tetrathiadicyclopenta[a,e]pentalene ring, a 911-fluorene ring, and the like.

In the aromatic ring which may have a substituent, the type of the substituent which may be contained in the aromatic ring is not particularly limited, and examples thereof include the substituent W. In a case where the aromatic ring has substituents, the number of substituents may be 1 or more (for example, 1 to 4 or the like).

In the present specification, the term “aromatic ring group” includes, for example, a group obtained by removing one or more hydrogen atoms (for example, 1 to 5 or the like) from the aromatic ring.

In the present specification, the term “aryl group” includes, for example, a group obtained by removing one hydrogen atom from a ring corresponding to an aromatic hydrocarbon ring among the above aromatic rings.

In the present specification, the term “heteroaryl group” includes, for example, a group obtained by removing one hydrogen atom from a ring corresponding to an aromatic heterocyclic ring among the above aromatic rings.

In the present specification, the term “arylene group” includes, for example, a group obtained by removing two hydrogen atoms from a ring corresponding to an aromatic hydrocarbon ring among the above aromatic rings.

In the present specification, the term “heteroarylene group” includes, for example, a group obtained by removing two hydrogen atoms from a ring corresponding to an aromatic heterocyclic ring among the above aromatic rings.

In an aromatic ring group which may have a substituent, an aryl group which may have a substituent, a heteroaryl group which may have a substituent, an arylene group which may have a substituent, and a heteroarylene group which may have a substituent, a kind of the substituents that these groups may have is not particularly limited, and examples thereof include a substituent W. In a case where these groups each of which may have a substituent have substituents, the number of substituents may be 1 or more (for example, 1 to 4 or the like).

In the present specification, in a case where a plurality of identical symbols indicating a kind or the number of groups are present in Formula (General Formula), which indicates a chemical structure, contents of these plurality of identical symbols indicating a kind or the number of groups are independent of each other, and the contents of the identical symbols may be the same or different from each other unless otherwise specified.

In the present specification, in a case where a plurality of identical groups (alkyl groups or the like) are present in one Formula (General Formula), which indicates a chemical structure, specific contents between these plurality of identical groups are independent of each other, and the specific contents between the plurality of identical groups may be the same or different from each other, unless otherwise specified.

A bonding direction of a divalent group (for example, —CO—O—) described in the present specification is not limited unless otherwise specified. For example, in a case where Y is —CO—O— in the compound represented by General Formula “X-Y-Z”, the compound may be “X—O—CO—Z” or may be “X—CO—O—Z”.

[Photoelectric conversion element] The photoelectric conversion element according to an embodiment of the present invention includes a conductive film, a photoelectric conversion film, and a transparent conductive film in this order, in which the photoelectric conversion film contains a compound represented by Formula (1) described later (hereinafter, referred to as a “specific compound”).

The mechanism capable of solving the above problems by adopting such a configuration of the photoelectric conversion element according to the embodiment of the present invention is not always clear, but the present inventors have presumed as follows.

It is presumed that in the specific compound, the indolenyl moiety (which means a moiety composed of a benzene ring having a substituent represented by B¹ and a nitrogen-containing 5-membered ring condensed with the benzene ring in Formula (1)) satisfies a predetermined condition, and thus the variation in dark current of the photoelectric conversion element is suppressed. Specifically, it is presumed that in a case where B¹ has a structure in which the HOMO energy calculated using a compound represented by Formula (B-1) described later as a model compound is less than −4.80 eV, the electron-withdrawing properties of the substituent contained in B¹ has an appropriate strength, and as a result, the variation in dark current of the photoelectric conversion element is suppressed.

In addition, it is presumed that in the specific compound, in a case where R³, R⁴, and R⁵ represent predetermined substituents, the specific compound tends to have a high phase-transition temperature, and as a result, the heat resistance of the photoelectric conversion element is improved.

Hereinafter, the fact that the variation in dark current is further suppressed and/or the heat resistance is more excellent is also referred to as an “effect of the present invention is more excellent”.

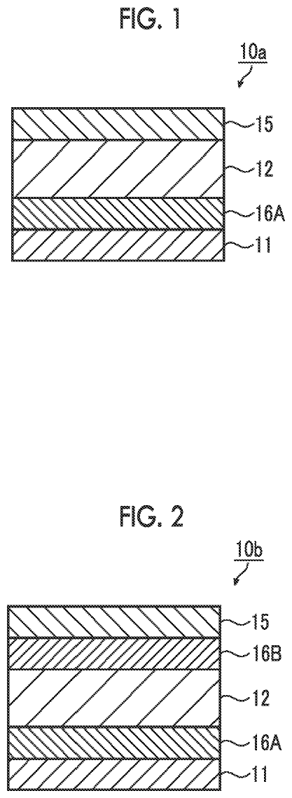

FIG. 1 is a schematic cross-sectional view of one embodiment of a photoelectric conversion element according to the embodiment of the present invention.

A photoelectric conversion element 10a illustrated in FIG. 1 has a configuration in which a conductive film (hereinafter, also referred to as a “lower electrode”) 11 functioning as a lower electrode, an electron blocking film 16A, a photoelectric conversion film 12 containing the specific compound described later, and a transparent conductive film (hereinafter, also referred to as an “upper electrode”) 15 functioning as an upper electrode are laminated in this order.

FIG. 2 illustrates a configuration example of another photoelectric conversion element. A photoelectric conversion element 10b illustrated in FIG. 2 has a configuration in which the electron blocking film 16A, the photoelectric conversion film 12, a positive hole blocking film 16B, and the upper electrode 15 are laminated on the lower electrode 11 in this order. The lamination order of the electron blocking film 16A, the photoelectric conversion film 12, and the positive hole blocking film 16B in FIGS. 1 and 2 may be appropriately changed according to the application and the characteristics.

In the photoelectric conversion element 10a (or 10b), it is preferable that light is incident on the photoelectric conversion film 12 through the upper electrode 15.

In a case where the photoelectric conversion element 10a (or 10b) is used, a voltage can be applied. In this case, it is preferable that the lower electrode 11 and the upper electrode 15 form a pair of electrodes, and a voltage is applied between the pair of electrodes.

The above-described voltage is preferably 1.0×10⁻⁵ to 1.0×10⁷ V/cm, and from the viewpoint of performance and power consumption, more preferably 1.0×10⁻⁴ to 1.0×10⁷ V/cm, and still more preferably 1.0×10⁻³ to 5.0×10⁶ V/cm.

Regarding a voltage application method, in FIGS. 1 and 2, it is preferable that the voltage is applied such that the electron blocking film 16A side is a cathode and the photoelectric conversion film 12 side is an anode. In a case where the photoelectric conversion element 10a (or 10b) is used as an optical sensor, or also in a case where the photoelectric conversion element 10a (or 10b) is incorporated in an imaging element, the voltage can be applied by the same method.

As described in detail below, the photoelectric conversion element 10a (or 10b) can be suitably applied to applications of the imaging element.

Hereinafter, the form of each layer constituting the photoelectric conversion element according to the embodiment of the present invention will be described in detail.

[Photoelectric conversion film] The photoelectric conversion film is a film containing a specific compound.

Hereinafter, the specific compound will be described in detail.

### <Compound Represented by Formula (1) (Specific Compound)>

The specific compound is the compound represented by Formula (1).

Formula (1) includes all geometric isomers that can be distinguished based on the C═C double bond (intended to mean the C═C double bond on the right side of the two double bonds specified in Formula (1)) constituted by a carbon atom to which R¹ bonds and a carbon atom adjacent thereto in Formula (1). That is, both the cis isomer and the trans isomer which are distinguished based on the C═C double bond are included in the compound represented by Formula (1).

In addition, Formula (1) includes all geometric isomers that can be distinguished based on the C═C double bond (intended to mean the C═C double bond on the left side of the two double bonds specified in Formula (1)) constituted by a carbon atom to which R² bonds and a carbon atom adjacent thereto in Formula (1). That is, both the cis isomer and the trans isomer which are distinguished based on the C═C double bond are included in the compound represented by Formula (1).

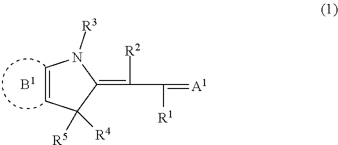

In Formula (1), R¹ represents a hydrogen atom or a substituent.

Examples of the substituent represented by R¹ include a group exemplified by the substituent W.

R¹ is preferably a hydrogen atom.

In Formula (1), R² represents a hydrogen atom or a substituent.

Examples of the substituent represented by R² include a group exemplified by the substituent W.

R² is preferably a hydrogen atom.

In Formula (1), R³ represents a linear or branched alkyl group which has a molecular weight of 160 or less and may have a substituent, a cycloalkyl group which may have a substituent, an aryl group which may have a substituent, or a heteroaryl group which may have a substituent.

Examples of the substituent which may be contained in the linear or branched alkyl group having a molecular weight of 160 or less, a cycloalkyl group, an aryl group, and a heteroaryl group, which is represented by R³ include the group exemplified by the substituent W.

The linear or branched alkyl group which has a molecular weight of 160 or less and may have a substituent, means that in a case where the linear or branched alkyl group has a substituent, the molecular weight including the substituent is 160 or less.

Particularly, R³ preferably represents a linear or branched alkyl group which has a molecular weight of 160 or less and may have a substituent, a cycloalkyl group which may have a substituent, or an aryl group which may have a substituent.

R⁴ and R⁵ each independently represent a substituent.

R⁴ and R⁵ may be bonded to each other to form a ring.

Examples of the substituent represented by R⁴ and R⁵ include a group exemplified by the substituent W.

Particularly, the substituent represented by R⁴ and R⁵ preferably represents a linear or branched alkyl group which may have a substituent, a cycloalkyl group which may have a substituent, an aryl group which may have a substituent, or a heteroaryl group which may have a substituent. Examples of the substituent which may be contained in the above-described alkyl group, a cycloalkyl group, an aryl group, and a heteroaryl group include the group exemplified by the substituent W.

In a case where R⁴ and R⁵ are bonded to each other to form a ring, the ring formed by bonding R⁴ and R⁵ to each other may be any of an aromatic ring or an alicyclic ring.

The alicyclic ring formed by bonding R⁴ and R⁵ to each other may be any of a monocyclic ring or a polycyclic ring (for example, 2 to 6 rings or the like). The alicyclic ring may be a spiro ring.

The number of carbon atoms of the alicyclic ring is not particularly limited, but is preferably 3 to 15, more preferably 3 to 10, still more preferably 3 or 6, and particularly preferably 5 or 6.

The alicyclic ring may be any of an aliphatic hydrocarbon ring or an aliphatic heterocyclic ring, but from the viewpoint that the effect of the present invention is more excellent, the alicyclic ring is preferably an aliphatic hydrocarbon ring.

Examples of the aliphatic heterocyclic ring include a ring having a ring structure in which a methylene group contained in the aliphatic hydrocarbon ring as a ring member atom is substituted with one or more selected from the group consisting of —O—, —S—, —NR^{A}—, —CO—, and —SO—, and the like. R^{A} represents a hydrogen atom or a substituent. Examples of the substituent represented by R^{A} include a group exemplified by the substituent W.

Particularly, the alicyclic ring formed by bonding R⁴ and R⁵ to each other is preferably a cycloalkane having 3 to 10 ring members (preferably 3 to 6 ring members and more preferably 5 or 6 ring members), in which a methylene group contained therein as a ring member atom may be substituted with one or more selected from the group consisting of —O—, —S—, —NR^{A}—, —CO—, and —SO—. Specific examples thereof include cyclopropane, cyclobutane, and cyclohexane.

In addition, the alicyclic ring formed by bonding R⁴ and R⁵ to each other may further have a substituent. The type of the substituent which may be contained in the alicyclic ring is not particularly limited, and examples thereof include the substituent W. In a case where the alicyclic ring has substituents, the number of substituents may be 1 or more (for example, 1 to 4 or the like).

Provided that in the compound represented by Formula (1), in a case where R³ represents a linear or branched alkyl group which has a molecular weight of 160 or less and may have a substituent, R⁴ and R⁵ each independently represent a cycloalkyl group which may have a substituent, an aryl group which may have a substituent, or a heteroaryl group which may have a substituent, or R⁴ and R⁵ are bonded to each other to form a ring containing no oxygen atom as a ring member atom.

In the compound represented by Formula (1), in a case where R³ represents a linear or branched alkyl group which has a molecular weight of 160 or less and may have a substituent, from the viewpoint that the effect of the present invention is more excellent, R⁴ and R⁵ are preferably bonded to each other to form a ring not including an oxygen atom as a ring member atom.

In addition, in the compound represented by Formula (1), it is preferable that R⁴ and R⁵ each independently represent a cycloalkyl group which may have a substituent, an aryl group which may have a substituent, or a heteroaryl group which may have a substituent, or R⁴ and R⁵ are bonded to each other to form a ring containing no oxygen atom as a ring member atom.

A¹ represents a group represented by Formula (A-1) or a group represented by Formula (A-2).

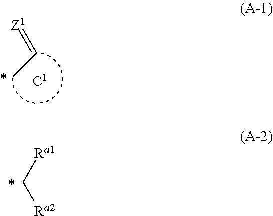

In Formula (A-1), * represents a bonding position.

Z¹ represents an oxygen atom, a sulfur atom, ═NR^{Z1}, or ═CR^{Z2}R^{Z3}

R^{Z1} represents a hydrogen atom or a substituent.

R^{Z2} and R^{Z3} each independently represent a cyano group, —SO₂R^{Z4}, —COOR^{Z5}, or —COR^{Z6}.

R^{Z4}, R^{Z5}, and R^{Z6} each independently represent an alkyl group which may have a substituent, an aryl group which may have a substituent, or a heteroaryl group which may have a substituent.

Examples of the substituents which are represented by R^{Z4}, R^{Z5}, and R^{Z6} and may be included in an alkyl group, an aryl group, and a heteroaryl group include the group exemplified by the substituent W.

Particularly, R^{Z2} and R^{Z3} are preferably a cyano group.

Z¹ preferably represents an oxygen atom.

In Formula (1), C¹ represents a ring which contains at least two carbon atoms and may have a substituent.

The two carbon atoms mean a carbon atom that is bonded to Z¹ specified in Formula (A-1), and a carbon atom that is adjacent to the carbon atom bonded to the Z¹ and is specified in Formula (A-1) (a carbon atom bonded with the carbon atom bonded to R¹ to form a double bond), and any carbon atom is an atom constituting C¹.

In addition, in the above ring, carbon atoms constituting the ring may be substituted with another carbonyl carbon (>C═O) and/or another thiocarbonyl carbon (>C═S). The other carbonyl carbon (>C═O) and the other thiocarbonyl carbon (>C═S) as used herein respectively mean a carbonyl carbon and a thiocarbonyl carbon each of which has a carbon atom other than the carbon atom bonded to Z¹ among the carbon atoms constituting the ring, as a constituent.

The carbon atoms of C¹ are preferably 3 to 30, more preferably 3 to 20, and still more preferably 3 to 15. The above-described carbon atoms are a number containing two carbon atoms specified in the formula.

C¹ may have a hetero atom, and examples thereof preferably include a nitrogen atom, a sulfur atom, an oxygen atom, a selenium atom, a tellurium atom, a phosphorus atom, a silicon atom, and a boron atom.

The number of hetero atoms in C¹ is preferably 0 to 10, more preferably 0 to 5, and still more preferably 0 to 2. The number of hetero atoms in which a carbon atom constituting the ring represented by C¹ is substituted by the carbonyl carbon (>C═O) or the thiocarbonyl carbon (>C═S) and introduced into the ring (the carbonyl carbon (>C═O) described herein includes the carbonyl carbon specified in Formula (A-1)), and the number of hetero atoms that a substituent of C¹ has is not included in the number of hetero atoms.

C¹ may have a substituent, and the substituent is preferably a halogen atom (preferably a chlorine atom), an alkyl group (may be any of linear, branched, or cyclic, having preferably 1 to 10 carbon atoms and more preferably 1 to 6 carbon atoms), an aryl group (having preferably 6 to 18 carbon atoms and more preferably 6 to 12 carbon atoms), a heteroaryl group (having preferably 5 to 18 carbon atoms and more preferably 5 to 6 carbon atoms), or a silyl group (examples thereof include an alkylsilyl group, the alkyl group in the alkylsilyl group may be linear, branched, or cyclic, and in addition, has preferably 1 to 4 carbon atoms and more preferably one carbon atom).

C¹ may or may not exhibit aromaticity.

C¹ may have a monocyclic structure or a fused ring structure, but is preferably a 5-membered ring, a 6-membered ring, or a fused ring containing at least any one of a 5-membered ring or a 6-membered ring. The number of rings forming the fused ring is preferably 2 to 4, and more preferably 2 to 3.

The ring represented by C¹ is usually preferably a ring used as an acidic nucleus (specifically, an acidic nucleus of a merocyanine coloring agent), and specific examples thereof are as follows:
(a) 1,3-dicarbonyl nuclei: for example, a 1,3-indandione nucleus, 1,3-cyclohexanedione, 5,5-dimethyl-1,3-cyclohexanedione, 1,3-dioxane-4,6-dione, and the like;
(b) pyrazolinone nuclei: examples thereof include 1-phenyl-2-pyrazolin-5-one, 3-methyl-1-phenyl-2-pyrazolin-5-one, 3-cyano-1-phenyl-2-pyrazolin-5-one, 3-trifluoromethyl-1-phenyl-2-pyrazolin-5-one, 1-(2-benzothiazolyl)-3-methyl-2-pyrazolin-5-one, and the like;
(c) isoxazolinone nuclei: for example, 3-phenyl-2-isoxazolin-5-one, 3-methyl-2-isoxazolin-5-one, and the like;
(d) oxindole nuclei: for example, 1-alkyl-2,3-hydro-2-oxindole, and the like;
(e) 2,4,6-trioxohexahydropyrimidine nuclei: for example, barbituric acid or 2-thibarbituric acid and derivatives thereof, and the like, and examples of the derivatives include 1-alkyl compounds such as 1-methyl and 1-ethyl, 1,3-dialkyl compounds such as 1,3-dimethyl, 1,3-diethyl, and 1,3-dibutyl, 1,3-diaryl compounds such as 1,3-diphenyl, 1,3-di(p-chlorophenyl), and 1,3-di(p-ethoxycarbonylphenyl), 1-alkyl-1-aryl compounds such as 1-ethyl-3-phenyl, 1,3-diheteroaryl compounds such as 1,3-di(2-pyridyl), and the like;
(f) 2-thio-2,4-thiazolidinedione nuclei: for example, rhodanine and derivatives thereof, and the like, and examples of the derivatives include 3-aklylrhodanine such as 3-methylrhodanine, 3-ethylrhodanine, and 3-allylrhodanine, 3-arylrhodanine such as 3-phenylrhodanine, 3-heteroarylrhodanine such as 3-(2-pyridyl)rhodanine, and the like;
(g) 2-thio-2,4-oxazolidinedione nuclei (2-thio-2,4-(3H,5H)-oxazoledione nuclei): for example, 3-ethyl-2-thio-2,4-oxazolidinedione, and the like;
(h) thianaphthenone nuclei: for example, 3(2H)-thianaphthenone-1,1-dioxide, and the like;
(i) 2-thio-2,5-thiazolidinedione nuclei: for example, 3-ethyl-2-thio-2,5-thiazolidinedione, and the like;
(j) 2,4-thiazolidinedione nuclei: for example, 2,4-thiazolidinedione, 3-ethyl-2,4-thiazolidinedione, 3-phenyl-2,4-thiazolidinedione, and the like;
(k) thiazoliin-4-one nuclei: for example, 4-thiazolinone, 2-ethyl-4-thiazolinone, and the like;
(l) 2,4-imidazolidinedione (hydantoin) nuclei: for example, 2,4-imidazolidinedione, 3-ethyl-2,4-imidazolidinedione, and the like;
(m) 2-thio-2,4-imidazolidinedione (2-thiohydantoin) nuclei: for example, 2-thio-2,4-imidazolidinedione, 3-ethyl-2-thio-2,4-imidazolidinedione, and the like;
(n) imidazolin-5-one nuclei: for example, 2-propylmercapto-2-imidazolin-5-one, and the like;
(o) 3,5-pyrazolidinedione nuclei: for example, 1,2-diphenyl-3,5-pyrazolidinedione, 1,2-dimethyl-3,5-pyrazolidinedione, and the like;
(p) benzothiophen-3(2H)-one nuclei: for example, benzothiophen-3(2H)-one, oxobenzothiophen-3(2H)-one, dioxobenzothiophen-3(2H)-one, and the like;
(q) indanone nuclei: examples thereof include 1-indanone, 3-phenyl-1-indanone, 3-methyl-1-indanone, 3,3-diphenyl-1-indanone, 3-(dicyanomethylidene)-1-indanone, 3,3-dimethyl-1-indanone, and the like;
(r) benzofuran-3-(2H)-one nucleus: for example, benzofuran-3-(2H)-one, and the like;
(s) 2,2-dihydrophenalene-1,3-dione nucleus, and the like.

C¹ may be a ring having a group represented by Formula (CX).

*1-L-Y-D-*2 (CX)

In Formula (CX), *1 represents a bonding position with the carbon atom in —C(═Z¹)-which is specified in Formula (A-1). *2 represents a bonding position with a carbon atom marked with * in Formula (A-1) (in other words, *2 represents a bonding position with a carbon atom bonded to the carbon atom, to which R¹ in Formula (1) is directly bonded to form a double bond).

In Formula (CX), L represents a single bond or —NR^{L}—.

R^{L} represents a hydrogen atom or a substituent.

Particularly, the substituent represented by R^{L} is preferably an alkyl group which may have a substituent, an aryl group which may have a substituent, or a heteroaryl group which may have a substituent.

Examples of the substituents which are represented by R^{L} and may be included in an alkyl group, an aryl group, and a heteroaryl group include the group exemplified by the substituent W.

L is preferably a single bond.

Y represents —CR^{Y1}═CR^{Y2}—, —CS—NR^{Y3}—, —CO—NR^{Y4}—, —CO—, —CS—, or —NR^{Y5}—, and among these, —CR^{Y1}═CR^{Y2}— is preferable.

R^{Y1} to R^{Y5} each independently represent a hydrogen atom or a substituent.

Particularly, R^{Y1} to R^{Y5} is each independently preferably an alkyl group which may have a substituent, an aryl group which may have a substituent, or a heteroaryl group which may have a substituent.

Examples of the substituents which are represented by R^{Y1} to R^{Y5} and may be included in an alkyl group, an aryl group, and a heteroaryl group include the group exemplified by the substituent W.

In addition, in a case where Y represents —CR^{Y1}═CR^{Y2}—, R^{Y1} and R^{Y2} are preferably linked to each other to form a ring, and R^{Y1} and R^{Y2} are more preferably linked to each other to form a benzene ring.

D represents a single bond, —O—, —S—, —SO₂—, —CO—, —CS—, —C(═NR^{D1})—, —C(═CR^{D2}R^{D3})—, —C(CN)═CR^{D4}—, or —N═CR^{D5}—, and among these, —O—, —S—, —SO₂—, —CO—, —CS—, —C(═NR^{D1})—, or —C(═CR^{D2}R^{D3})— is preferable.

R^{D1} represents a hydrogen atom or a substituent.

The type of the substituent represented by R^{D1} is not particularly limited, and examples thereof include a group exemplified by the substituent W. Particularly, an alkyl group which may have substituents, an aryl group which may have substituents, or a heteroaryl group which may have substituents are preferable.

Examples of the substituents which are represented by R^{D1} and may be included in an alkyl group, an aryl group, and a heteroaryl group include the group exemplified by the substituent W.

R^{D1} is more preferably a hydrogen atom.

R^{D2} and R^{D3} each independently represent a cyano group, —SO₂R^{D6}, —COOR^{D7}, or —COR^{D8}. R^{D6}, R^{D7}, and R^{D8} each independently represent an alkyl group which may have a substituent, an aryl group which may have a substituent, or a heteroaryl group which may have a substituent. Examples of the substituents which are represented by R^{D6}, R^{D7}, and R^{D8} and may be included in an alkyl group, an aryl group, and a heteroaryl group include the group exemplified by the substituent W.

Particularly, R^{D2} and R^{D3} are preferably a cyano group.

R^{D4} to R^{D5} each independently represent a cyano group or an alkyl group which may have a substituent.

Examples of the substituents which are represented by R^{D4} and R^{D5} and may be included in an alkyl group include the group exemplified by the substituent W. The substituent is preferably a fluorine atom.

The combination of L, Y, and D described above is preferably a combination of -L-Y-D-, which is bonded to two carbon atoms specified in Formula (A-1) to form a ring that is a 5-membered ring or a 6-membered ring. However, as described above, the 5-membered ring or the 6-membered ring may be fused with a different ring (preferably a benzene ring) to form a fused ring structure.

Examples of the group represented by Formula (CX) include a group represented by Formula (CX1), *1-NR^{L}—CS—NR^{Y3}—CO-*2, *1-NR^{L}—CO—NR^{Y4}—CO-*2, *1—NR^{L}—CO—C(CN)═CR^{D4}-*2, *1-NR^{L}—NR^{Y5}—CO-*2, and *1-NR^{Y5}—N═CR^{D5}-*2. The R^{L}, R^{Y3} to R^{Y5}, R^{D4}, and R^{D5} are as described above.

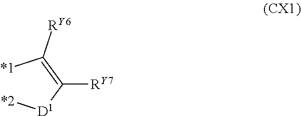

In Formula (CX1), R^{Y6} and R^{Y7} each independently represent a hydrogen atom or a substituent. In addition, R^{Y6} and R^{Y7} may be linked to each other to form a ring.

R^{Y6} and R^{Y7} are preferably linked to each other to form a ring, and R^{Y6} and R^{Y7} are more preferably linked to each other to form a benzene ring.

The benzene ring formed by R^{Y6} and R^{Y7} further preferably has a substituent. As the substituent, a halogen atom is preferable, and a chlorine atom or a fluorine atom is more preferable.

In addition, substituents included in the benzene ring formed by R^{Y6} and R^{Y7} may be further linked to each other to form a ring. For example, substituents that the above-described benzene ring formed by R^{Y6} and R^{Y7} has may be further linked to each other to form a benzene ring.

*¹, *², and D¹ in Formula (CX1) each have the same definitions as *¹, *², and D in Formula (CX) described above, and the suitable embodiments thereof are also the same. Particularly, D¹ is preferably —O—, —S—, —SO₂—, —CO—, —CS—, —C(═NR^{D1})—, or —C(═CR^{D2}R^{D3})—.

Particularly, the group represented by Formula (CX) is still more preferably a group represented by Formula (CX2).

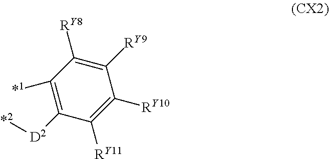

In Formula (CX2), R^{Y8} to R^{Y11} each independently represent a hydrogen atom or a substituent. Particularly, R^{Y8} to R^{Y11} are each independently preferably a hydrogen atom or a halogen atom, and more preferably a hydrogen atom, a chlorine atom, or a fluorine atom, and still more preferably a hydrogen atom or a chlorine atom.

R^{Y8} and R^{Y9} may be bonded to each other to form a ring, R^{Y9} and R^{Y10} may be bonded to each other to form a ring, and R^{Y10} and R^{Y11} may be bonded to each other to form a ring. The ring formed by linking R^{Y8} and R^{Y9}, R^{Y9} and R^{Y10}, and R^{Y10} and R^{Y11} is preferably a benzene ring. Among these, R^{Y9} and R^{Y10} are preferably bonded to each other to form a ring, and the ring formed by linking R^{Y9} and R^{Y10} to each other is preferably a benzene ring. The ring formed by linking R^{Y9} and R^{Y10} to each other may be further substituted with a substituent (preferably a halogen atom).

*¹, *², and D² in Formula (CX2) each have the same definitions as *¹, *², and D in Formula (CX) described above, and the suitable embodiments thereof are also the same. Particularly, D² is preferably —O—, —S—, —SO₂—, —CO—, —CS—, —C(═NR^{D1})—, or —C(═CR^{D2}R^{D3})—.

Particularly, the group represented by Formula (A-1) is preferably a group represented by Formula (A-3).

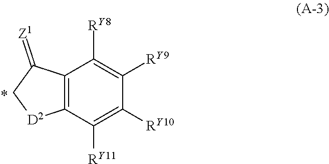

In Formula (A-3), * represents a bonding position.

Z¹ in Formula (A-3) has the same definition as Z¹ in Formula (1), and the suitable embodiments thereof are also the same.

D², R^{Y8} to R^{Y11} in Formula (A-3) each have the same definitions as D², R^{Y8} to R^{Y11} in Formula (CX2), and the suitable embodiments thereof are also the same.

In Formula (A-2), * represents a bonding position.

R^{a1} and R^{a2} each independently represent a cyano group, —SO₂R^{b1}, —COOR^{b2}, or —COR^{b3}.

R^{b1}, R^{b2}, and R^{b3} each independently represent an alkyl group which may have a substituent, an aryl group which may have a substituent, or a heteroaryl group which may have a substituent.

Examples of the substituents which are represented by R^{b1}, R^{b2}, and R^{b3} and may be included in an alkyl group, an aryl group, and a heteroaryl group include the group exemplified by the substituent W.

In Formula (1), A¹ preferably represents a group represented by Formula (A-1) and more preferably represents a group represented by Formula (A-3).

In Formula (B-1), B¹ represents a benzene ring having a substituent. Provided that B¹ satisfies Condition BX.

### <Condition BX>

The compound represented by Formula (B-1) has HOMO energy of less than −4.80 eV, which is obtained by performing structure-optimization calculation according to density-functional calculation B3LYP/6-31G(d) using quantum chemical calculation software Gaussian09.

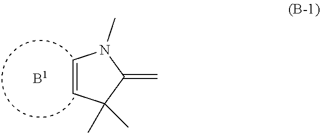

B¹ in Formula (B-1) represents a benzene ring having a substituent.

B¹ in Formula (B-1) is the same as B¹ in Formula (1).

In addition, in Formula (B-1), a substituent attached to a nitrogen atom is a methyl group, a carbon atom adjacent to the nitrogen atom (a carbon atom on a side not constituting a ring member atom of B¹) is >C═CH₂, and two substituents attached to a carbon atom adjacent to the carbon atom adjacent to the nitrogen atom (a carbon atom on a side not constituting a ring member atom of B¹) are each a methyl group.

In a case where the compound represented by Formula (1) represents, for example, the Compound (D-1), the compound represented by Formula (B-1) which is a model compound corresponds to the compound represented by Formula (D-B-1).

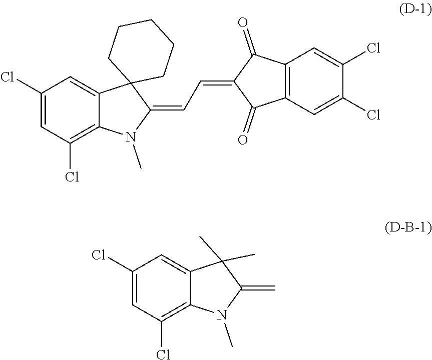 [SMILES: CN1/C(=C/C=C2C(=O)c3cc(Cl)c(Cl)cc3C2=O)C2(CCCCC2)c2cc(Cl)cc(Cl)c21 ; C=C1N(C)c2c(Cl)cc(Cl)cc2C1(C)C]

The compound represented by Formula (B-1) in which B¹ represents a non-substituted benzene ring, has HOMO energy of −4.80 eV, which is obtained by performing structure-optimization calculation according to density-functional calculation B3LYP/6-31G(d) using quantum chemical calculation software Gaussian09. Therefore, in a case where the substituent contained in the benzene ring represented by B¹ exhibits electron-withdrawing properties as a whole, the HOMO energy measured by the above-described method tends to be less than −4.80 eV. Here, a case where the substituent contained in the benzene ring represented by B¹ exhibits electron-withdrawing properties as a whole means that in a case where the benzene ring represented by B¹ has only one substituent, the substituent is an electron-withdrawing group. In addition, in a case where the benzene ring represented by B¹ has two or more substituents, this means that the sum of the strengths of the respective electron-withdrawing properties of the two or more substituents exhibits electron-withdrawing properties. Therefore, even in a case where the benzene ring represented by B¹ has an electron-donating substituent and an electron-withdrawing substituent, the HOMO energy measured by the above-described method tends to be less than −4.80 eV as long as the substituents exhibit electron-withdrawing properties as a whole.

The number of substituents contained in the benzene ring represented by B₁ is not particularly limited, and examples thereof include 1 to 4.

The substituent contained in the benzene ring represented by B¹ is preferably an electron-withdrawing group having a Hammett's sigma-para value (σp value) of more than 0.05, and examples thereof include a fluorine atom, a chlorine atom, a perfluoroalkyl group, a cyano group, a nitro group, an acyl group, an alkylsulfonyl group, an arylsulfonyl group, a sulfamoyl group, and a sulfinyl group. These electron-withdrawing groups may further have a substituent.

Hammett's rule is an experimental rule proposed by L. P. Hammett in 1935 to quantitatively discuss the effects of substituents on the reaction or equilibrium of a benzene derivative, and the validity thereof is widely accepted today. Substituent constants as a values obtained by the Hammett rule are an σp value and an am value, and these values can be found in many general books. For example, the detail can be found in “Lange's Handbook of Chemistry” 12th edition, edited by J. A. Dean, 1979 (McGraw-Hill), “Kagaku no Ryoiki (Journal of Japanese Chemistry) special edition” vol. 122, pp. 96-103, 1979 (Nankodo), and “Chem. Rev.” vol. 91, pp. 165-195, 1991.

Specific examples thereof include a fluorine atom (0.06), a chlorine atom (0.23), a perfluoroalkyl group (—CF₃: 0.54), a cyano group (0.66), an alkoxycarbonyl group (—COOMe: 0.45), an aryloxycarbonyl group (—COOPh: 0.44), an alkylcarbonyl group (—COMe: 0.50), an arylcarbonyl group (—COPh: 0.43), an alkylsulfonyl group (—SO₂Me: 0.72), and an arylsulfonyl group (—SO₂Ph: 0.68). The values in the parentheses are representative σp values of the substituents extracted from “Chem. Rev.” vol. 91, pp. 165-195, 1991. In addition, Me represents a methyl group, and Ph represents a phenyl group.

From the viewpoint that the effect of the present invention is more excellent, the HOMO energy measured by the above-described method is preferably −4.90 eV or less, more preferably −5.00 eV or less, and still more preferably −5.10 eV or less. A lower limit value thereof is, for example, −5.60 eV or more.

In addition, it is preferable that the substituent contained in the benzene ring represented by B¹ does not include a substituent having a Hammett's substituent constant σp of 0.05 or less.

Specific examples of the specific compound corresponding to the compound represented by Formula (1) will be shown below, but the present invention is not limited thereto.

In a case where the specific compounds exemplified below were applied to Formula (1), the specific compounds exemplified below include all geometric isomers that can be distinguished based on the C═C double bond (intended to mean the C═C double bond on the right side of the two double bonds specified in Formula (1)) constituted by a carbon atom to which R¹ bonds and a carbon atom adjacent thereto. That is, both the cis isomer and the trans isomer which are distinguished based on the C═C double bond are included in the specific compounds exemplified below, respectively.

In addition, in a case where the specific compounds exemplified below were applied to Formula (1), the specific compounds exemplified below include all geometric isomers that can be distinguished based on the C═C double bond (intended to mean the C═C double bond on the left side of the two double bonds specified in Formula (1)) constituted by a carbon atom to which R² bonds and a carbon atom adjacent thereto. That is, both the cis isomer and the trans isomer which are distinguished based on the C═C double bond are included in the specific compounds exemplified below, respectively.

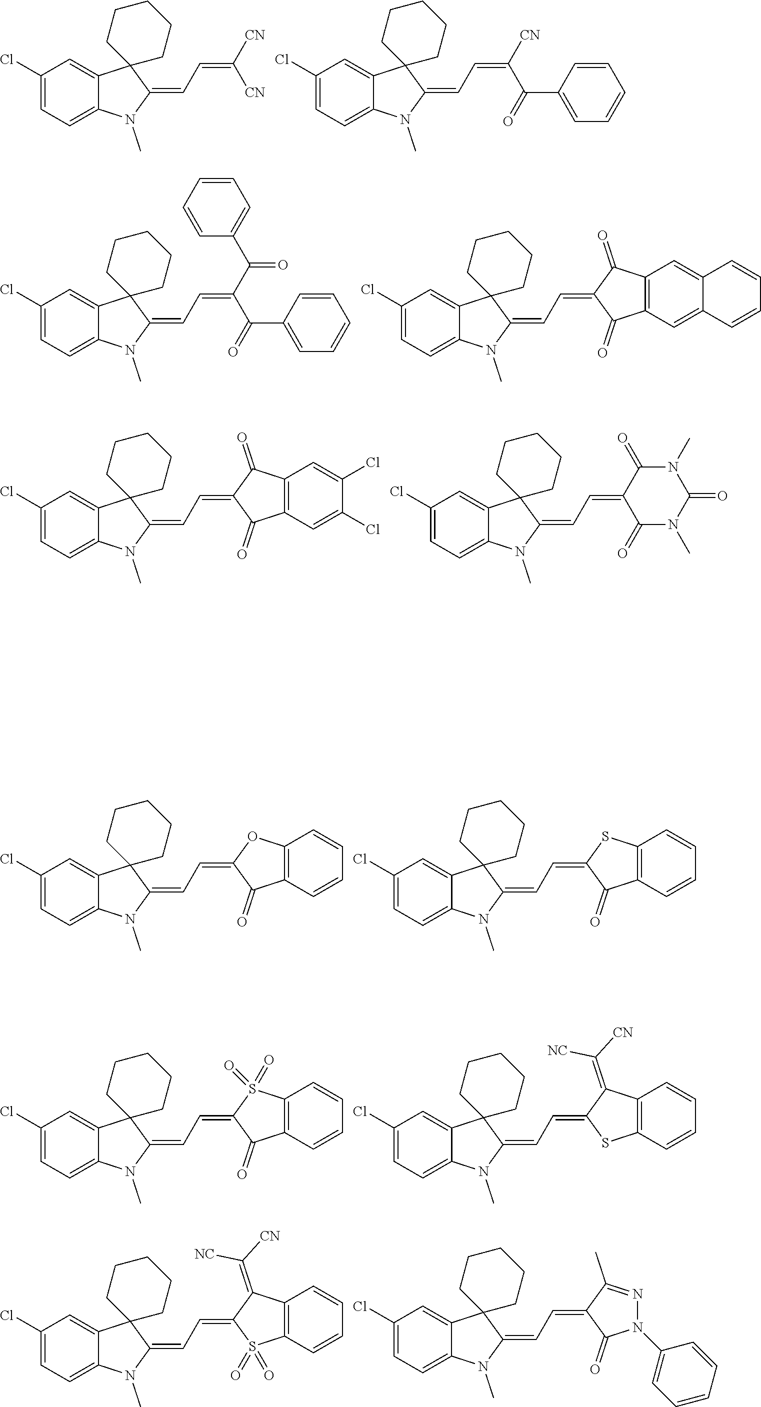

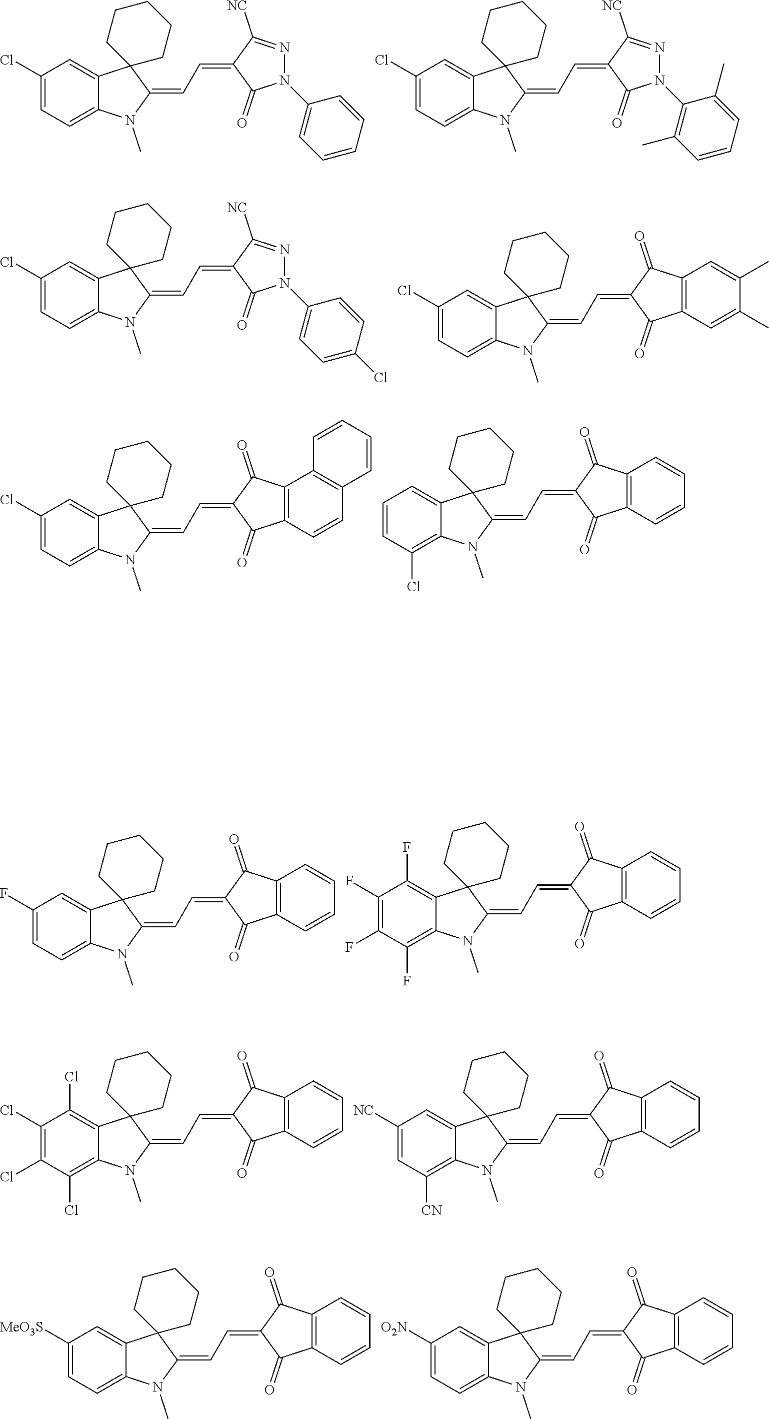

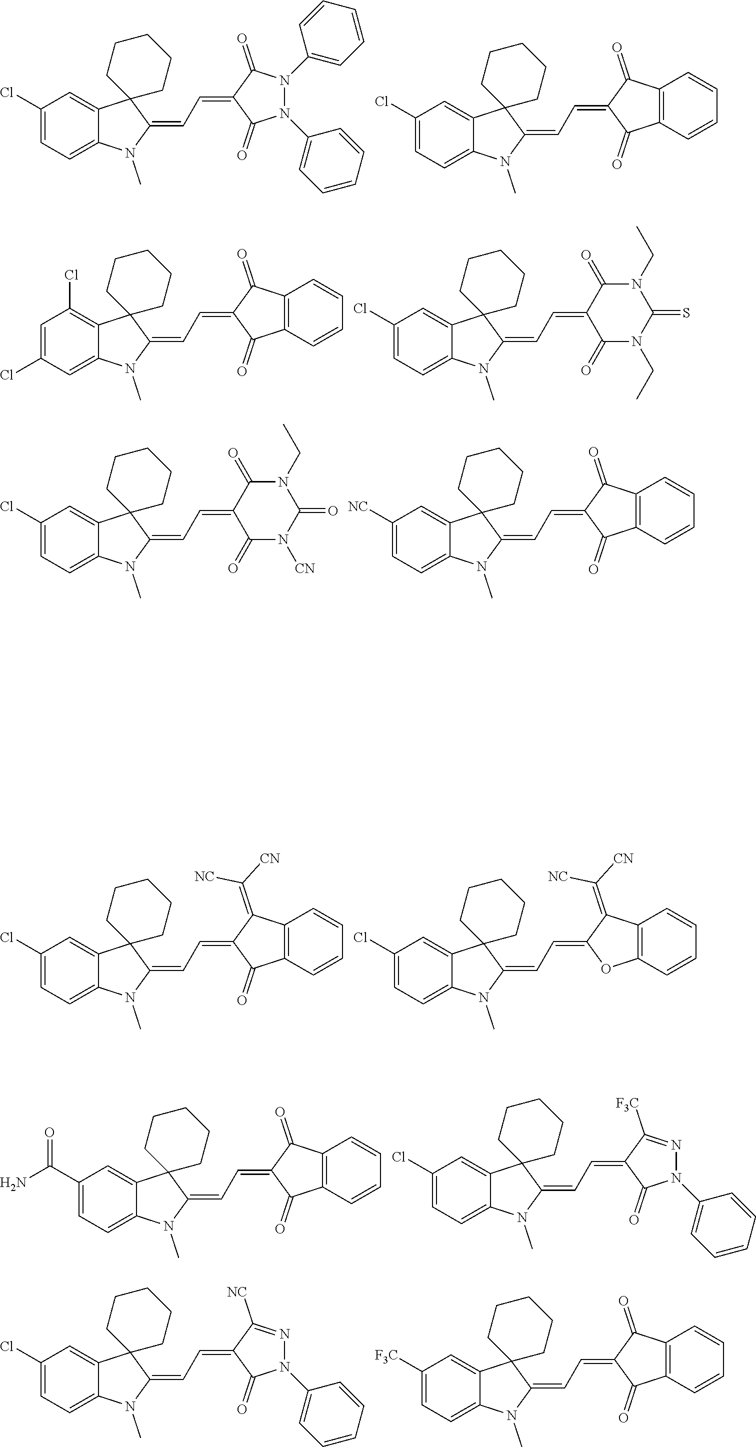

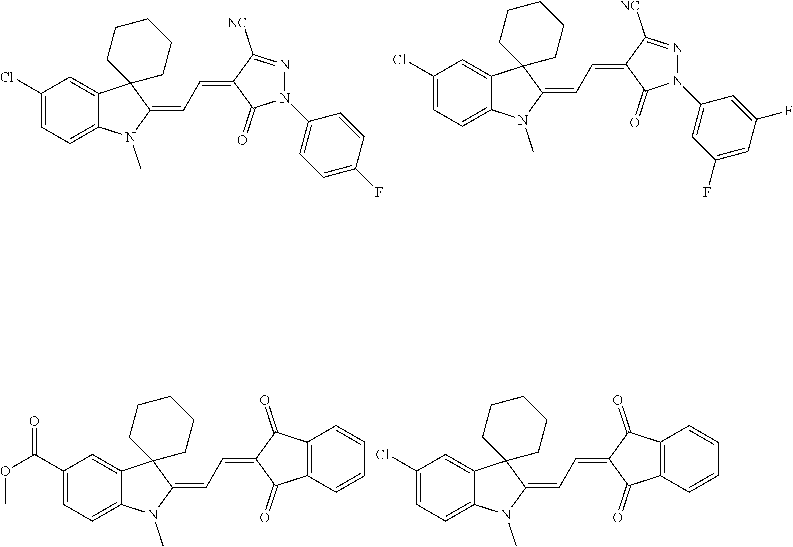

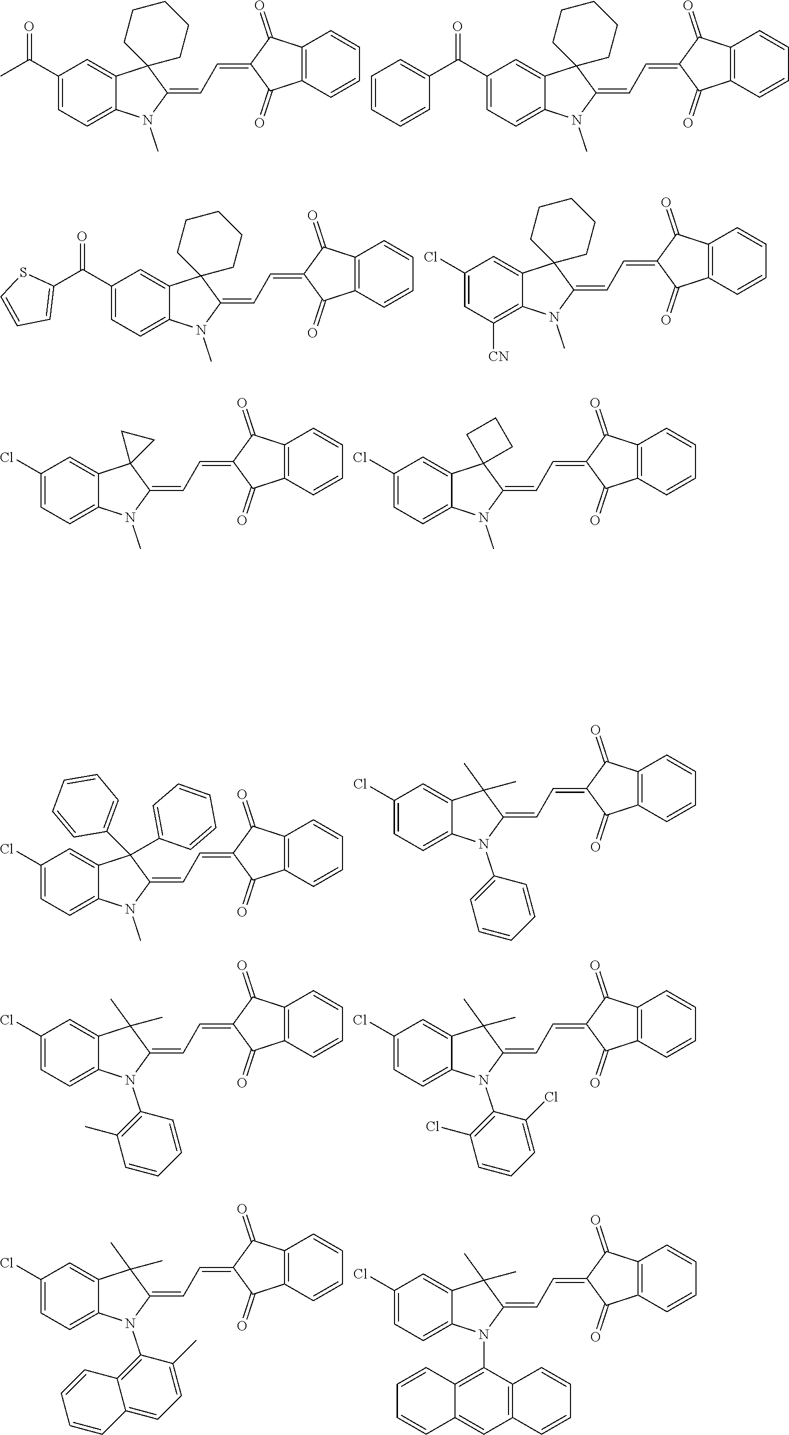

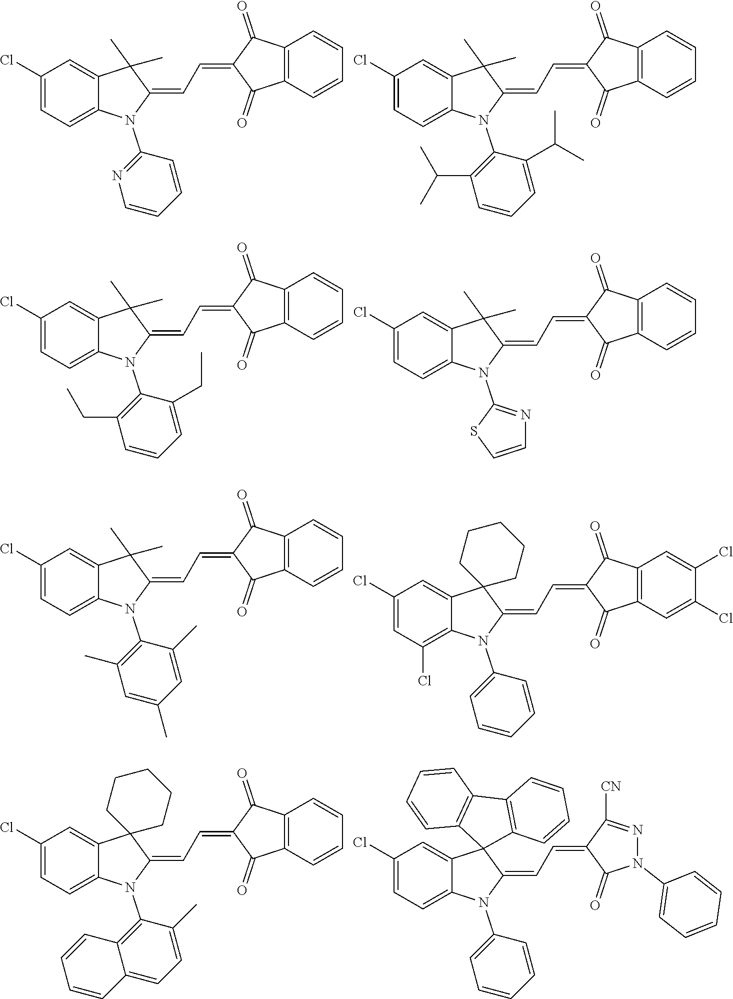

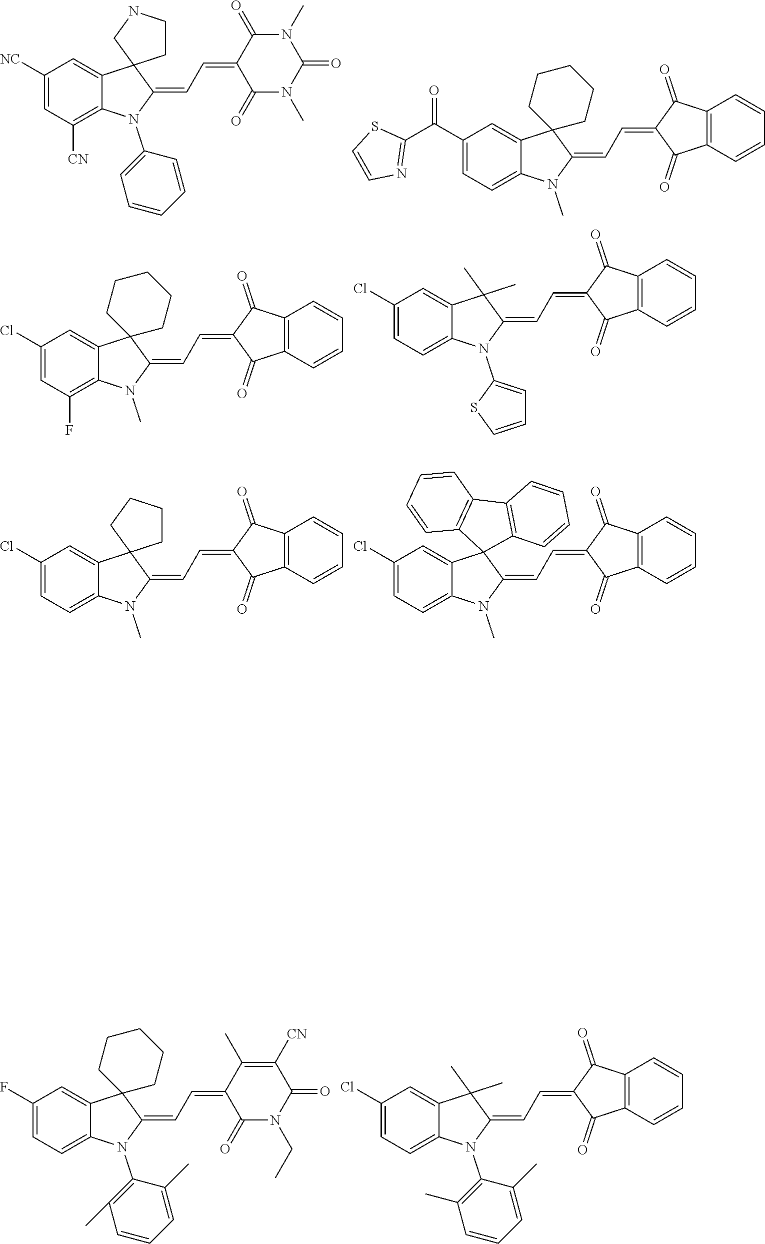

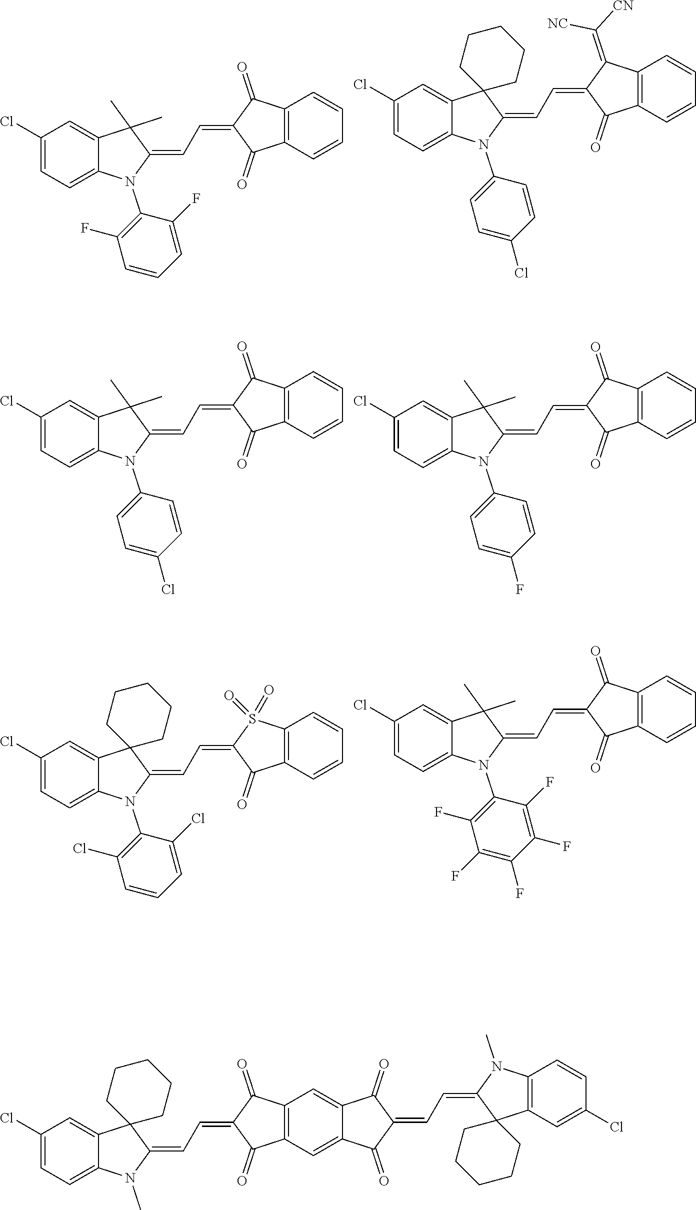

The molecular weight of the specific compound is not particularly limited, but from the viewpoint of excellent manufacturing suitability, is preferably 400 to 800, more preferably 400 to 700, and still more preferably 400 to 600.

In a case where the molecular weight is in the above-described range, it is presumed that the sublimation temperature of the specific compound becomes low, and the photoelectric conversion efficiency is excellent also in a case where a photoelectric conversion film is formed at a high speed.

The specific compound is particularly useful as a material of the photoelectric conversion film used for the imaging element, the optical sensor, or a photoelectric cell. The specific compound can also be used as a coloring material, a liquid crystal material, an organic semiconductor material, a charge transport material, a pharmaceutical material, and a fluorescent diagnostic material.

The specific compound is preferably a compound in which an ionization potential in a single film is −6.0 to −5.0 eV from the viewpoints of stability in a case of using the compound as the p-type organic semiconductor and matching of energy levels between the compound and the n-type organic semiconductor.

The maximum absorption wavelength of the specific compound is preferably in a range of 400 to 700 nm, more preferably in a range of 450 to 650 nm, and still more preferably in a range of 450 to 600 nm.

The maximum absorption wavelength is a value measured in a solution state (solvent: chloroform) by an absorption spectrum of the coloring agent being adjusted to a concentration having an absorbance of about 0.5 to 1. However, in a case where the coloring agent is not dissolved in chloroform, a value measured by using the coloring agent in which the coloring agent is vapor-deposited and formed into a film state is defined as a maximum absorption wavelength of the coloring agent.

The specific compound may be purified as necessary.

A purification method of the specific compound is not particularly limited, but sublimation purification is preferably employed.

The purity of the specific compound after sublimation purification (for example, the purity measured by high performance liquid chromatography (HPLC) or gas chromatograph (GC)) is not particularly limited, but is preferably 95% or more, more preferably 98% or more, and still more preferably 99% or more.

Before the specific compound is purified by sublimation, the specific compound may be purified by another method. For example, the specific compound is preferably purified by using silica gel column chromatography, gel permeation chromatography (GPC), reslurry washing, reprecipitation, an adsorbent such as activated carbon, or recrystallization.

The purity of the specific compound before sublimation purification (for example, the purity measured by HPLC or GC) is not particularly limited, but is preferably 95% or more, more preferably 98% or more, and still more preferably 99% or more.

A solvent (recrystallizing solvent) used in the recrystallization purification is not particularly limited, and examples thereof include methanol, ethanol, isopropanol, butanol, toluene, xylene, anisole, 1,2-dimethoxybenzene, tetralin, chlorobenzene, dichlorobenzene, hexane, heptane, octane, acetonitrile, benzonitrile, acetic acid, chloroform, dichloromethane, ethyl acetate, butyl acetate, tetrahydrofuran, 4-methyltetrahydropyran, cyclopentylmethyl ether, and other solvents.

The recrystallizing solvent may be a mixed solution in which a plurality of solvents are mixed.

The amount of a residual solvent contained in a crude product containing the specific compound to be subjected to the sublimation purification is not particularly limited, but the amount of the residual solvent is preferably 10% by mol or less, more preferably 5% by mol or less, and still more preferably 2% by mol or less with respect to the total molar amount of the specific compound in the crude product.

Impurities including elements (for example, Li, Na, K, Mg, Ca, Al, Si, P, Sn, transition metal elements, or other elements) that do not constitute the specific compound contained in the crude product containing the specific compound to be subjected to the sublimation purification are not particularly limited, but the impurities are preferably 1000 mass ppm or less, more preferably 100 mass ppm or less, and still more preferably 10 mass ppm or less with respect to the total mass of the crude product.

Examples of the method for measuring the elements include an ICP (high frequency inductively coupled plasma) emission analysis method.

The specific compound can be synthesized by a known method.

The purity of raw materials used to synthesize the specific compound (for example, the purity measured by HPLC or GC), including intermediates, to improve the purity of the specific compound is not particularly limited, but is preferably 97% or more, more preferably 98% or more, and still more preferably 99% or more.

In a case where the purity of the commercially available raw materials or synthetic intermediates is low, those purified by a known method may be used.

The specific compound may be used alone or in combination of two or more kinds thereof.

A content of the specific compound in the photoelectric conversion film (=film thickness of specific compound in terms of single layer/film thickness of photoelectric conversion film×100) is preferably 15% to 75% by volume, more preferably 20% to 60% by volume, and still more preferably 25% to 50% by volume.

<n-Type Organic Semiconductor>

The photoelectric conversion film preferably contains the n-type organic semiconductor in addition to the specific compound.

The n-type organic semiconductor is a compound different from the specific compound.

The n-type organic semiconductor is an acceptor-property organic semiconductor material (a compound), and refers to an organic compound having a property of easily accepting an electron. That is, the n-type organic semiconductor refers to an organic compound having a large electron affinity of two organic compounds used in contact with each other. That is, any organic compound having an electron accepting property can be used as the acceptor type organic semiconductor.

Examples of the n-type organic semiconductor include fullerenes selected from the group consisting of a fullerene and derivatives thereof, fused aromatic carbocyclic compounds (for example, a naphthalene derivative, an anthracene derivative, a phenanthrene derivative, a tetracene derivative, a pyrene derivative, a perylene derivative, and a fluoranthene derivative); a heterocyclic compound having a 5- to 7-membered ring having at least one selected from the group consisting of a nitrogen atom, an oxygen atom, and a sulfur atom (for example, pyridine, pyrazine, pyrimidine, pyridazine, triazine, quinoline, quinoxaline, quinazoline, phthalazine, cinnoline, isoquinoline, pteridine, acridine, phenazine, phenanthroline, tetrazole, pyrazole, imidazole, and thiazole); polyarylene compounds; fluorene compounds; cyclopentadiene compounds; silyl compounds; 1,4,5,8-naphthalenetetracarboxylic acid anhydride; 1,4,5,8-naphthalenetetracarboxylic acid anhydride imide derivative and oxadiazole derivatives; anthraquinodimethane derivatives; diphenylquinone derivatives; bathocuproine, bathophenanthroline, and derivatives thereof, triazole compounds; a distyrylarylene derivative; a metal complex having a nitrogen-containing heterocyclic compound as a ligand; a silole compound; and compounds disclosed in paragraphs [0056] to [0057] of JP2006-100767A.

The n-type organic semiconductor (compound) is preferably fullerenes selected from the group consisting of a fullerene and derivatives thereof.

Examples of the fullerenes include a fullerene C60, a fullerene C70, a fullerene C76, a fullerene C78, a fullerene C80, a fullerene C82, a fullerene C84, a fullerene C90, a fullerene C96, a fullerene C240, a fullerene C540, and a mixed fullerene.

Examples of the fullerene derivatives include compounds in which a substituent is added to the above fullerenes. The substituent is preferably an alkyl group, an aryl group, or a heterocyclic group. As the fullerene derivative, the compounds described in JP2007-123707A are preferable.

An organic coloring agent may be used as the n-type organic semiconductor.

Examples of the organic coloring agent include a cyanine coloring agent, a styryl coloring agent, a hemicyanine coloring agent, a merocyanine coloring agent (including zeromethine merocyanine (simple merocyanine)), a rhodacyanine coloring agent, an allopolar coloring agent, an oxonol coloring agent, a hemioxonol coloring agent, a squarylium coloring agent, a croconium coloring agent, an azamethine coloring agent, a coumarin coloring agent, an arylidene coloring agent, an anthraquinone coloring agent, a triphenylmethane coloring agent, an azo coloring agent, an azomethine coloring agent, a metallocene coloring agent, a fluorenone coloring agent, a flugide coloring agent, a perylene coloring agent, a phenazine coloring agent, a phenothiazine coloring agent, a quinone coloring agent, a diphenylmethane coloring agent, a polyene coloring agent, an acridine coloring agent, an acridinone coloring agent, a diphenylamine coloring agent, a quinophthalone coloring agent, a phenoxazine coloring agent, a phthaloperylene coloring agent, a dioxane coloring agent, a porphyrin coloring agent, a chlorophyll coloring agent, a phthalocyanine coloring agent, a subphthalocyanine coloring agent, and a metal complex coloring agent.

The molecular weight of the n-type organic semiconductor is preferably 200 to 1200, and more preferably 200 to 900.

It is also preferable that the n-type organic semiconductor is colorless or has a maximum absorption wavelength and/or an absorption waveform close to that of the specific compound. As specific numerical values, it is preferable that the maximum absorption wavelength of the n-type organic semiconductor is in a range of 400 nm or less or 500 to 600 nm.

It is preferable that the photoelectric conversion film has a bulk hetero structure formed in a state in which the specific compound and the n-type organic semiconductor are mixed. The bulk hetero structure refers to a layer in which the specific compound and the n-type organic semiconductor are mixed and dispersed in the photoelectric conversion film. The photoelectric conversion film having the bulk hetero structure can be formed by either a wet method or a dry method. The bulk hetero structure is described in detail in, for example, paragraphs [0013] and [0014] of JP2005-303266A.

In a case where the photoelectric conversion film contains the n-type organic semiconductor, a content of the n-type organic semiconductor in the photoelectric conversion film (=film thickness of n-type organic semiconductor in terms of single layer/film thickness of photoelectric conversion film×100) is preferably 15% to 75% by volume, more preferably 20% to 60% by volume, and still more preferably 25% to 50% by volume.

The n-type organic semiconductor material may be used alone, or two or more thereof may be used in combination.

In addition, in a case where the n-type organic semiconductor material includes fullerenes, a content of the fullerenes to a total content of the n-type organic semiconductor material (=film thickness of fullerenes in terms of single layer/total film thickness of n-type organic semiconductor materials in terms of single layer×100) is preferably 50% to 100% by volume, and more preferably 80% to 100% by volume.

The fullerenes may be used alone, or two or more thereof may be used in combination.

The difference in electron affinity between the specific compound and the n-type organic semiconductor is preferably 0.1 eV or more.

From the viewpoint of responsiveness of the photoelectric conversion element, the content of the specific compound to the total content of the specific compound and the n-type organic semiconductor (=film thickness in terms of single layer of specific compound/(film thickness in terms of single layer of specific compound+film thickness in terms of single layer of n-type organic semiconductor)×100) is preferably 20% to 80% by volume, and more preferably 40% to 80% by volume.

Also, in a case where the photoelectric conversion film further contains a p-type organic semiconductor described below, the content of the specific compound (=film thickness in terms of single layer of specific compound/(film thickness in terms of single layer of specific compound+film thickness in terms of single layer of n-type organic semiconductor+film thickness in terms of single layer of p-type organic semiconductor)×100) is preferably 15% to 75% by volume, and more preferably 30% to 75% by volume.

Also, in a case where the photoelectric conversion film further contains a coloring agent described below, the content of the specific compound (=film thickness in terms of single layer of specific compound/(film thickness in terms of single layer of specific compound+film thickness in terms of single layer of n-type organic semiconductor+film thickness in terms of single layer of coloring agent)×100) is preferably 15% to 75% by volume, and more preferably 30% to 75% by volume.

Also, in a case where the photoelectric conversion film further contains a p-type organic semiconductor described below and a coloring agent described below, the content of the specific compound (=film thickness in terms of single layer of specific compound/(film thickness in terms of single layer of specific compound+film thickness in terms of single layer of n-type organic semiconductor+film thickness in terms of single layer of p-type organic semiconductor+film thickness in terms of single layer of coloring agent)×100) is preferably 15% to 75% by volume, and more preferably 30% to 75% by volume.

It is preferable that the photoelectric conversion film is substantially formed of the specific compound, the n-type organic semiconductor, the p-type organic semiconductor included as desired, and the coloring agent included as desired. The term “substantially” means that a total content of the specific compound, the n-type organic semiconductor, the p-type organic semiconductor included as desired, and the coloring agent included as desired is 90% to 100% by volume (preferably 95% to 100% by volume, and more preferably 99% to 100% by volume) with respect to a total mass of the photoelectric conversion film.

<p-Type Organic Semiconductor>

The photoelectric conversion film preferably contains the p-type organic semiconductor in addition to the specific compound.

The p-type organic semiconductor is a compound different from the specific compound.

The p-type organic semiconductor is a donor organic semiconductor material (a compound), and refers to an organic compound having a property of easily donating an electron. That is, the p-type organic semiconductor means an organic compound having a smaller ionization potential in a case where two organic compounds are used in contact with each other.

The p-type organic semiconductor may be used alone, or two or more kinds thereof may be used in combination.

Examples of the p-type organic semiconductor include triarylamine compounds (for example, N, N′-bis (3-methylphenyl)-(1,1′-biphenyl)-4,4′-diamine (TPD), 4,4′-bis [N-(naphthyl)-N-Phenyl-amino] biphenyl (a-NPD), compounds disclosed in paragraphs [0128] to [0148] of JP2011-228614A, compounds disclosed in paragraphs [0052] to [0063] of JP2011-176259A, compounds disclosed in paragraphs [0119] to [0158] of JP2011-225544A, compounds disclosed in paragraphs [0044] to [0051] of JP2015-153910A, and compounds disclosed in paragraphs [0086] to [0090] of JP2012-94660A), pyrazoline compounds, styrylamine compounds, hydrazone compounds, polysilane compounds, thiophene compounds (for example, a thienothiophene derivative, a dibenzothiophene derivative, a benzodithiophene derivative, a dithienothiophene derivative, a [1]benzothieno[3,2-b]thiophene (BTBT) derivative, a thieno [3,2-f. 4,5-f] bis [1] benzothiophene (TBBT) derivative, compounds disclosed in paragraphs [0031] to [0036] of JP2018-014474A, compounds disclosed in paragraphs [0043] to [0045] of WO2016-194630A, compounds disclosed in paragraphs [0025] to [0037], and [0099] to [0109] of WO2017-159684A, compounds disclosed in paragraphs [0029] to [0034] of JP2017-076766A, compounds disclosed in paragraphs [0015] to [0025] of WO2018-207722A, compounds disclosed in paragraphs [0045] to [0053] of JP2019-054228A, compounds disclosed in paragraphs [0045] to [0055] of WO2019-058995A, compounds disclosed in paragraphs [0063] to [0089] of WO2019-081416A, compounds disclosed in paragraphs [0033] to [0036] of JP2019-80052A, compounds disclosed in paragraphs [0044] to [0054] of WO2019-054125A, compounds disclosed in paragraphs [0041] to [0046] of WO2019-093188A), compounds in paragraphs [0034] to [0037] of JP2019-050398A, compounds in paragraphs [0033] to [0036] of JP2018-206878A, compounds in paragraph [0038] of JP2018-190755A, compounds in paragraphs [0019] to [0021] of JP2018-026559A, compounds in paragraphs [0031] to [0056] of JP2018-170487A, compounds in paragraphs [0036] to [0041] of JP2018-078270A, compounds in paragraphs [0055] to [0082] of JP2018-166200A, compounds in paragraphs [0041] to [0050] of JP2018-113425A, compounds in paragraphs [0044] to [0048] of JP2018-85430A, compounds in paragraphs [0041] to [0045] of JP2018-056546A, compounds in paragraphs [0042] to [0049] of JP2018-046267A, compounds in paragraphs [0031] to [0036] of JP2018-014474A, compounds disclosed in paragraphs [0036] to [0046] of WO2018-016465A, compounds disclosed in paragraphs [0045] to [0048] of JP2020-010024A, and the like), a cyanine compound, an oxonol compound, a polyamine compound, an indole compound, a pyrrole compound, a pyrazole compound, a polyarylene compound, a fused aromatic carbocyclic compound (for example, a naphthalene derivative, an anthracene derivative, a phenanthrene derivative, a tetracene derivative, a pentacene derivative, a pyrene derivative, a perylene derivative, a fluoranthene derivative, and the like), a porphyrin compound, a phthalocyanine compound, a triazole compound, an oxadiazole compound, an imidazole compound, a polyarylalkane compound, a pyrazolone compound, an amino-substituted chalcone compound, an oxazole compound, a fluorenone compound, a silazane compound, and a metal complex having nitrogen-containing heterocyclic compounds as ligands.

Examples of the p-type organic semiconductor include compounds having an ionization potential smaller than that of the n-type organic semiconductor, and in a case where this condition is satisfied, the organic dyes exemplified as the n-type organic semiconductor can be used.

The compounds that can be used as the p-type semiconductor compound are exemplified below.

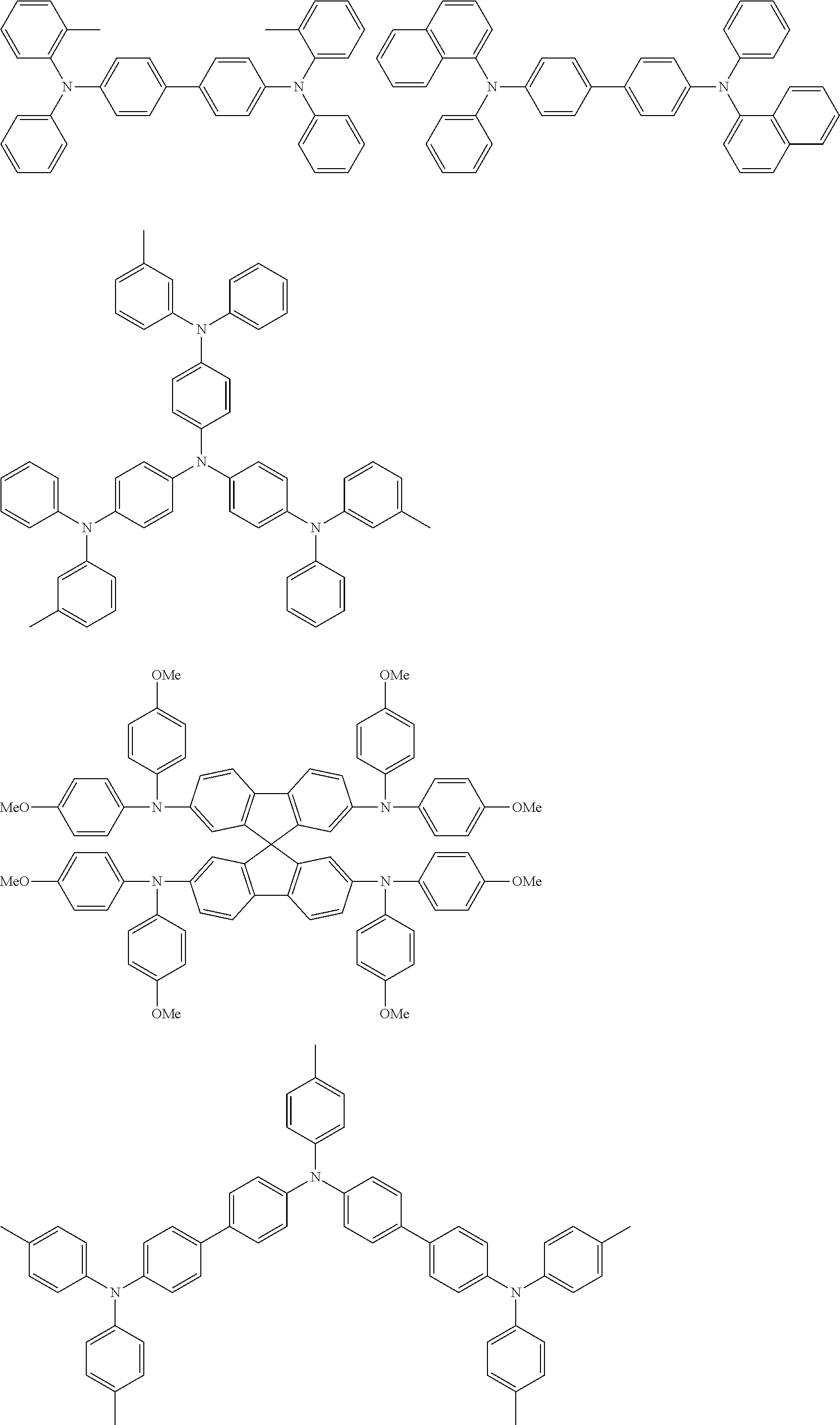

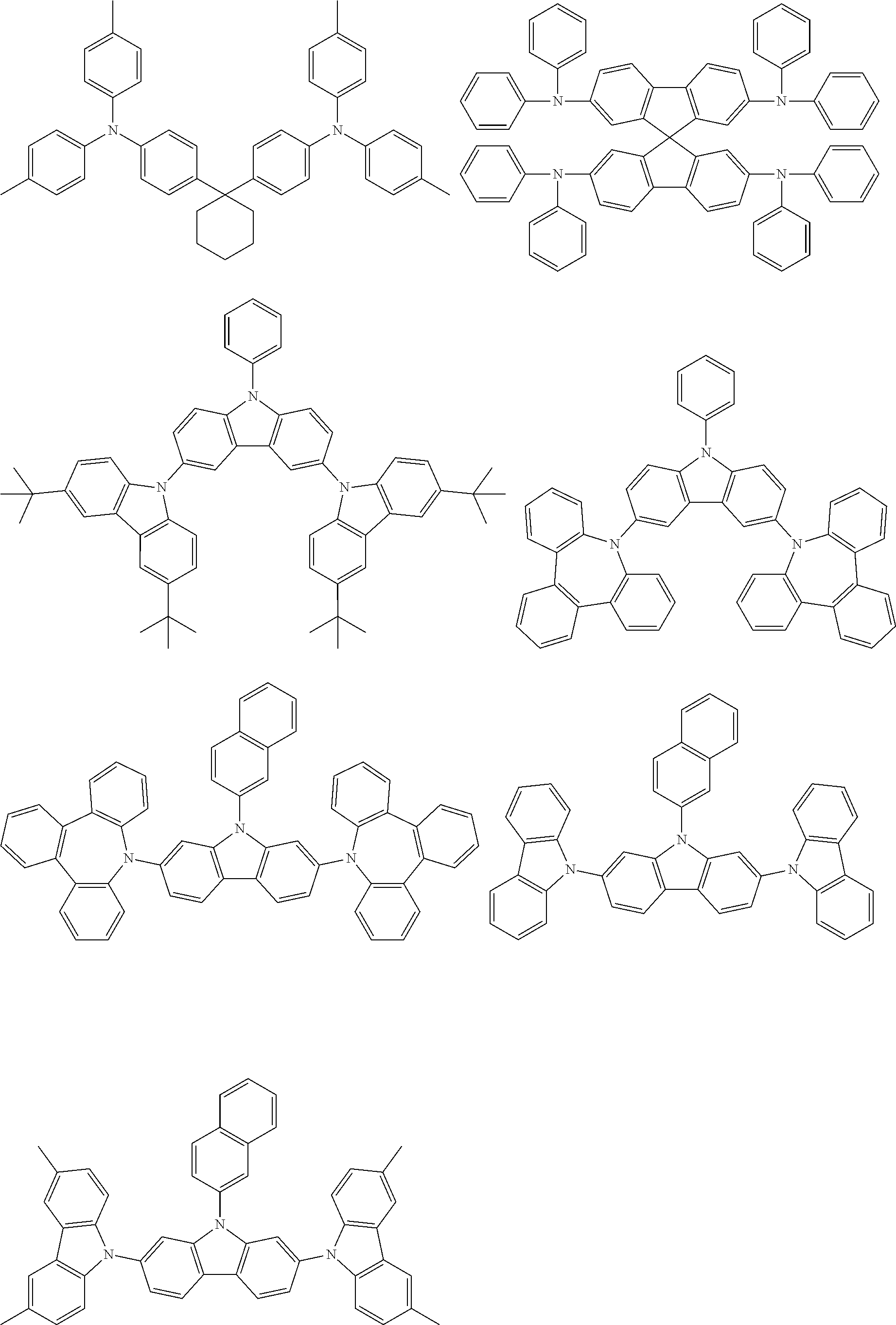

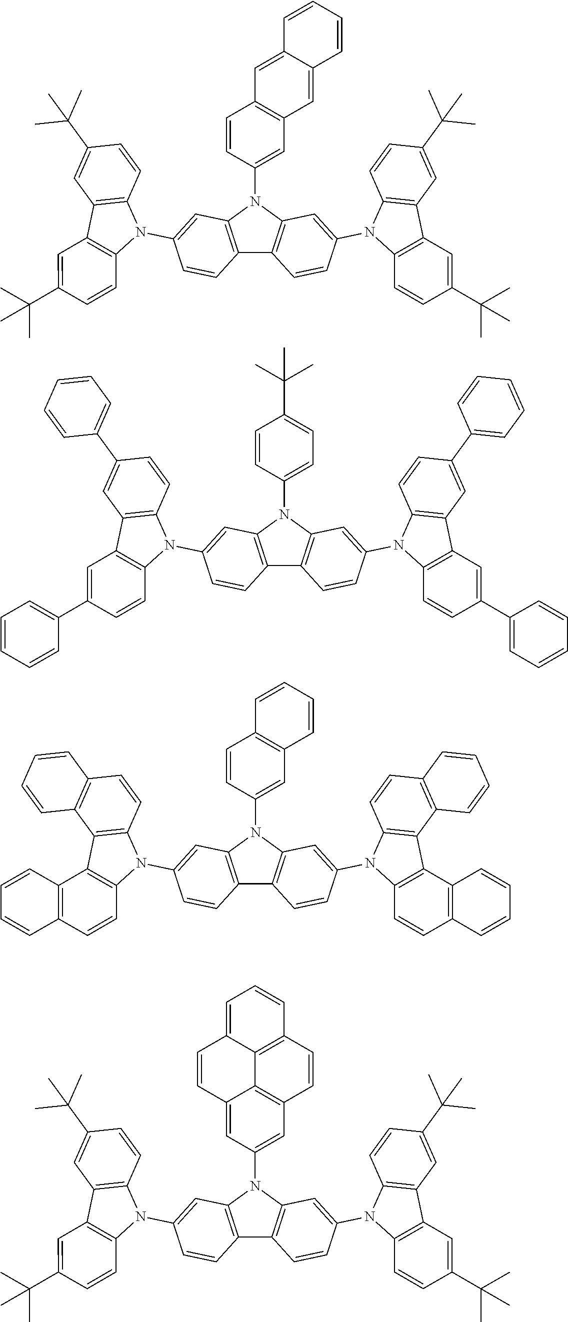

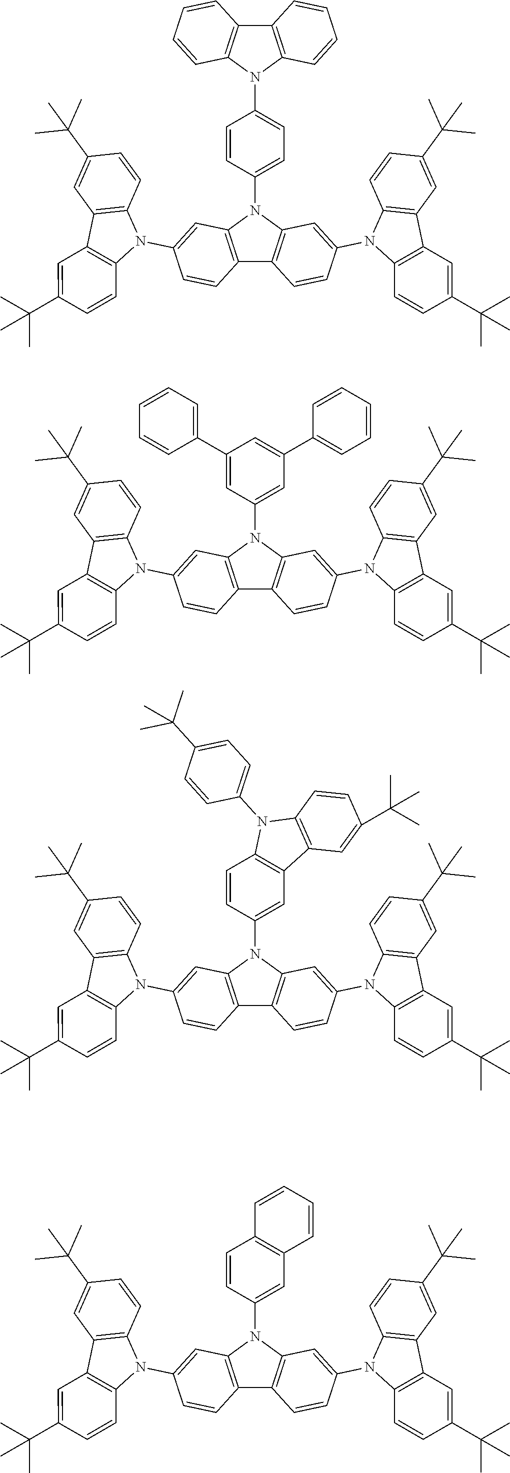

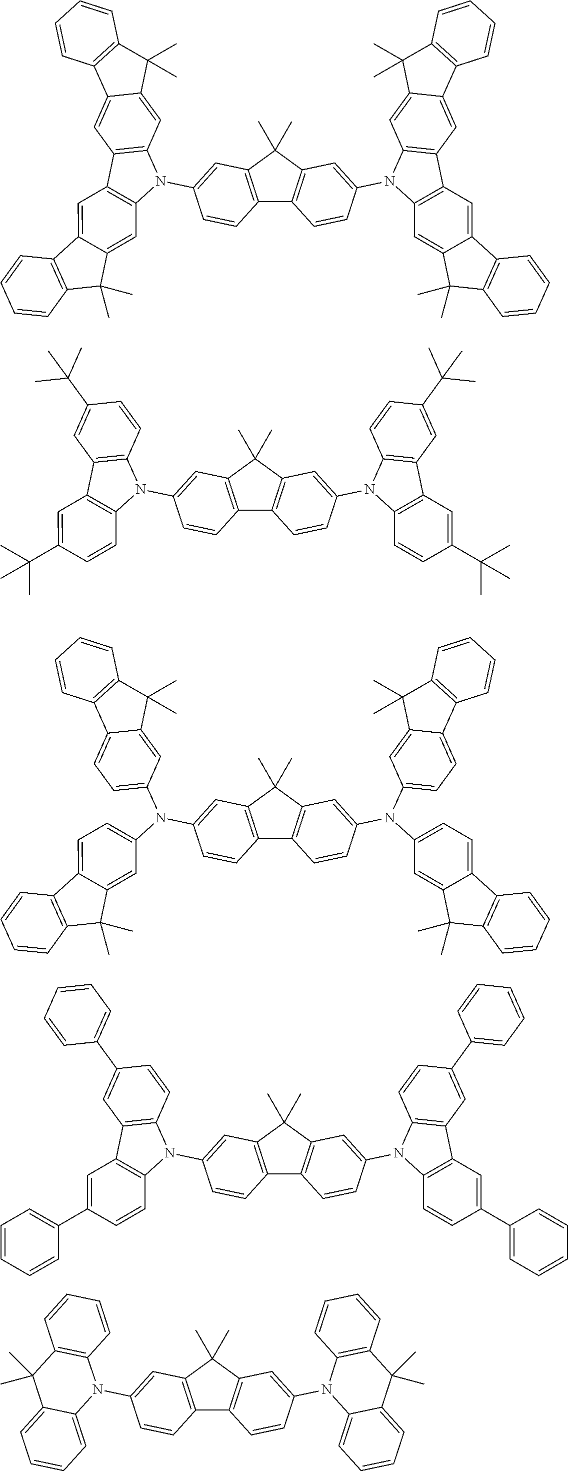

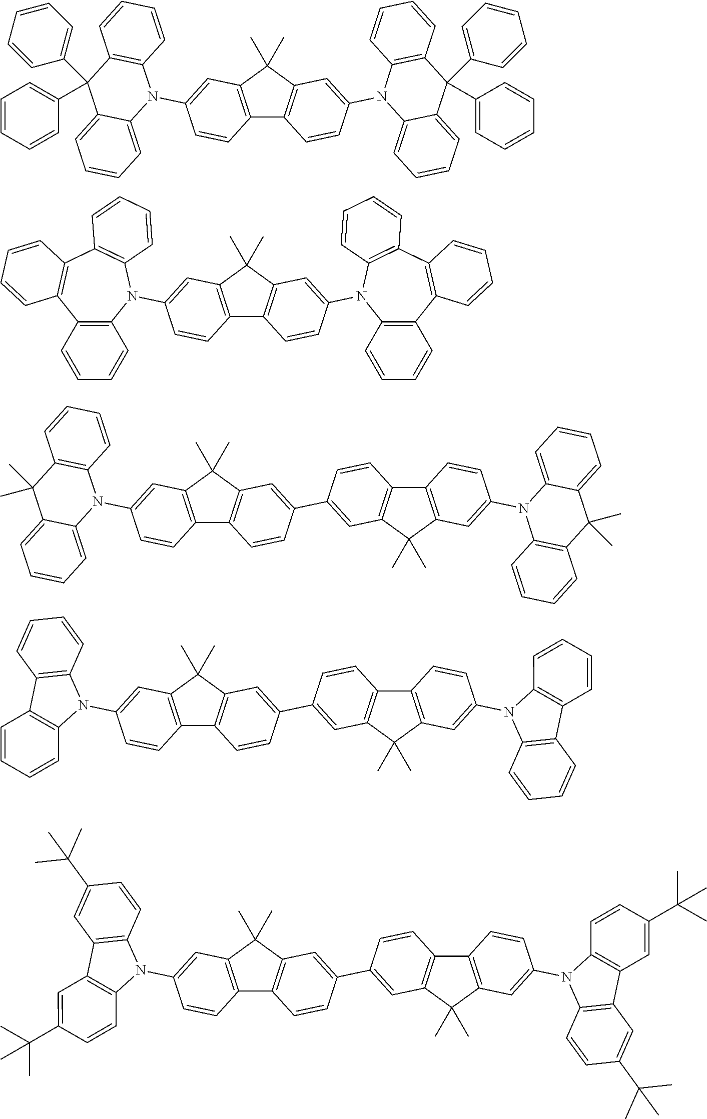

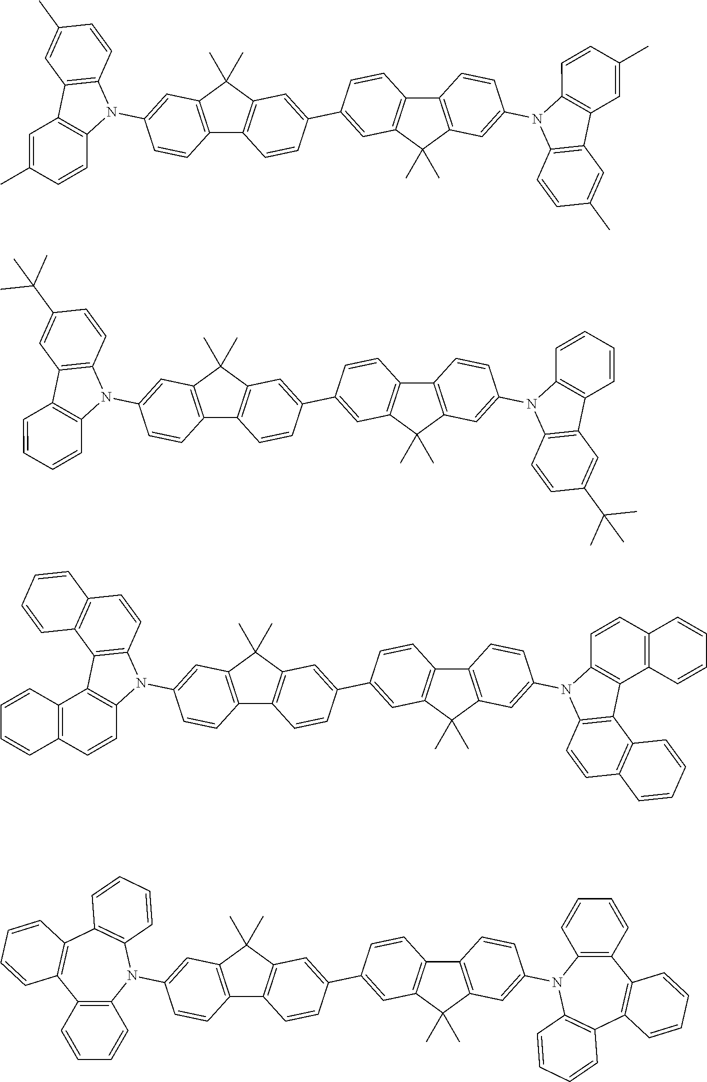

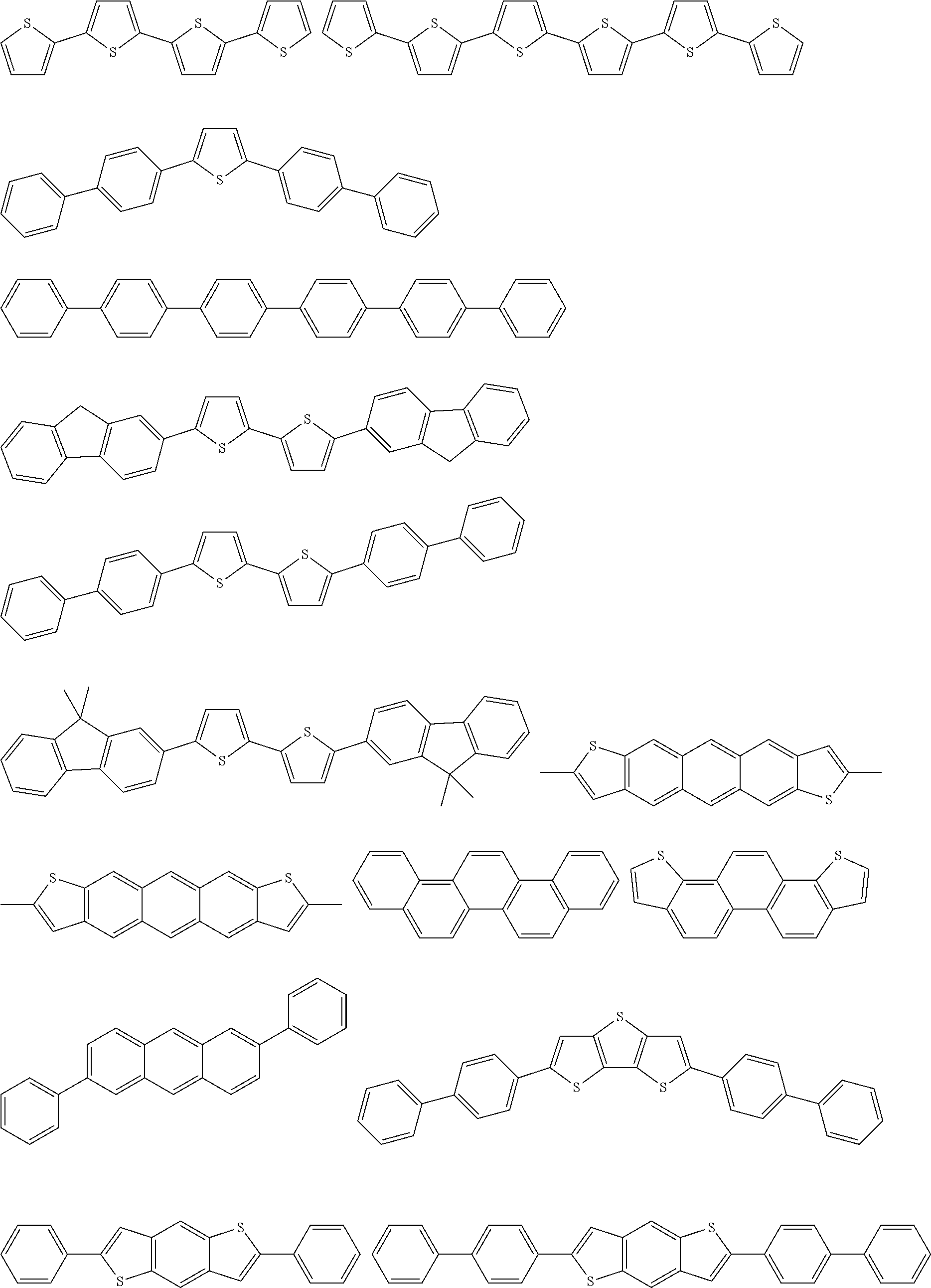

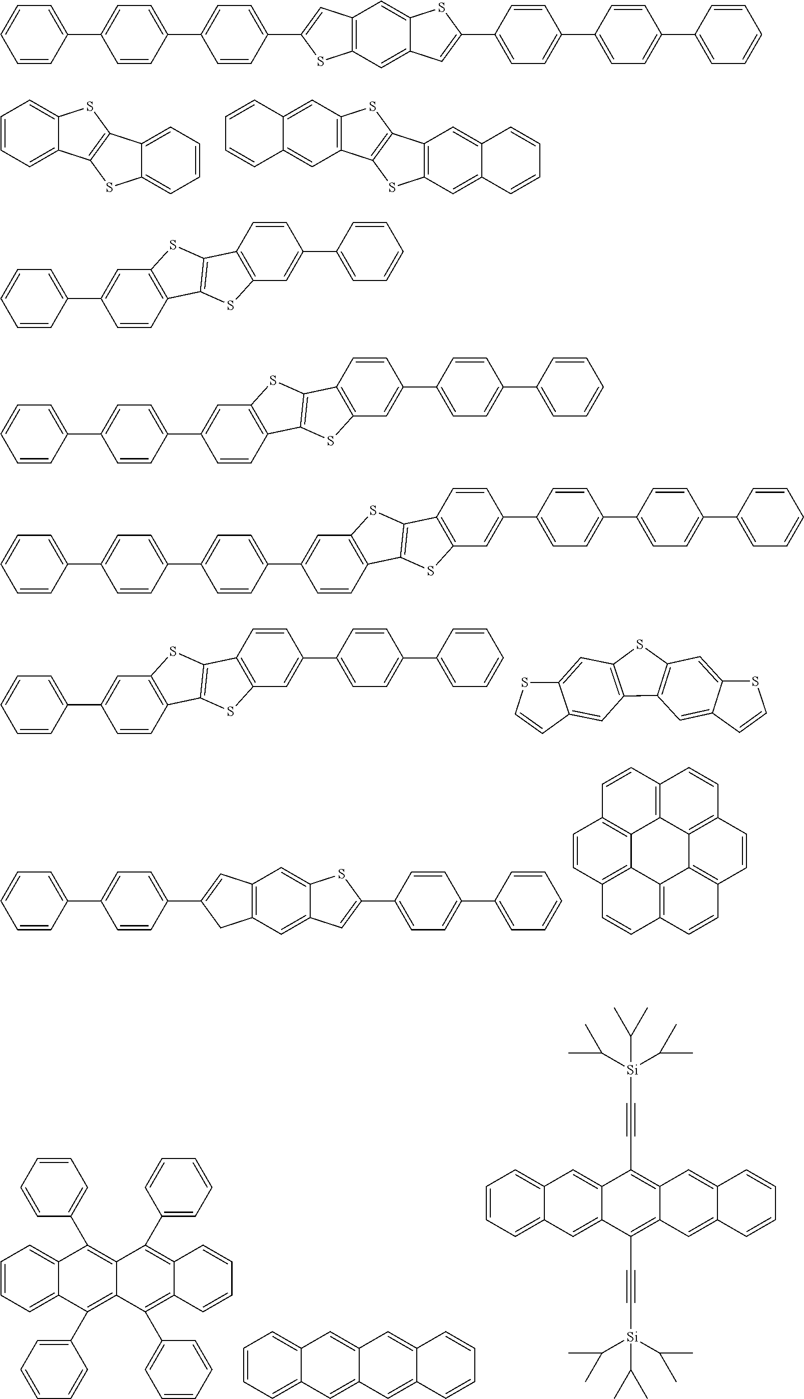

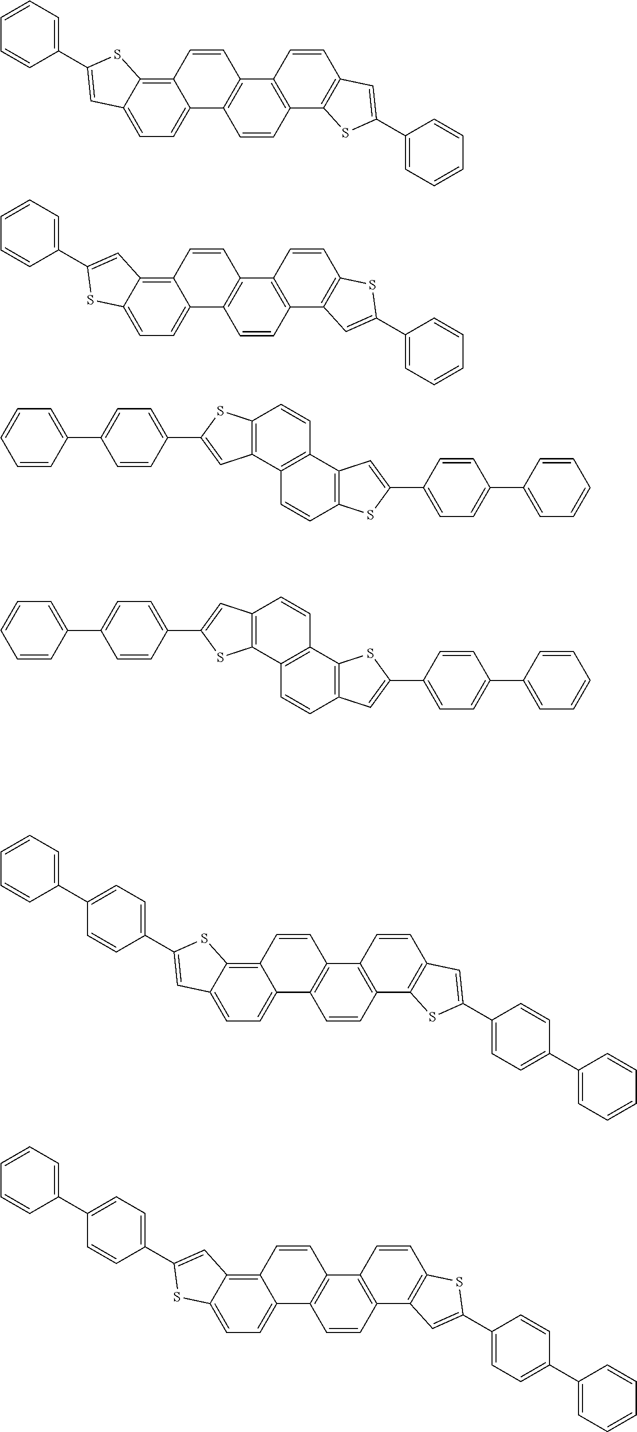 [SMILES: c1ccc(-c2cc3ccc4c5ccc6c(ccc7cc(-c8ccccc8)sc76)c5ccc4c3s2)cc1 ; c1ccc(-c2cc3c(ccc4c3ccc3c5ccc6sc(-c7ccccc7)cc6c5ccc43)s2)cc1 ; c1ccc(-c2ccc(-c3cc4c(ccc5c6cc(-c7ccc(-c8ccccc8)cc7)sc6ccc45)s3)cc2)cc1 ; c1ccc(-c2ccc(-c3cc4ccc5c(ccc6cc(-c7ccc(-c8ccccc8)cc7)sc65)c4s3)cc2)cc1 ; c1ccc(-c2ccc(-c3cc4ccc5c6ccc7c(ccc8cc(-c9ccc(-c%10ccccc%10)cc9)sc87)c6ccc5c4s3)cc2)cc1 ; c1ccc(-c2ccc(-c3cc4c(ccc5c4ccc4c6ccc7sc(-c8ccc(-c9ccccc9)cc8)cc7c6ccc54)s3)cc2)cc1]

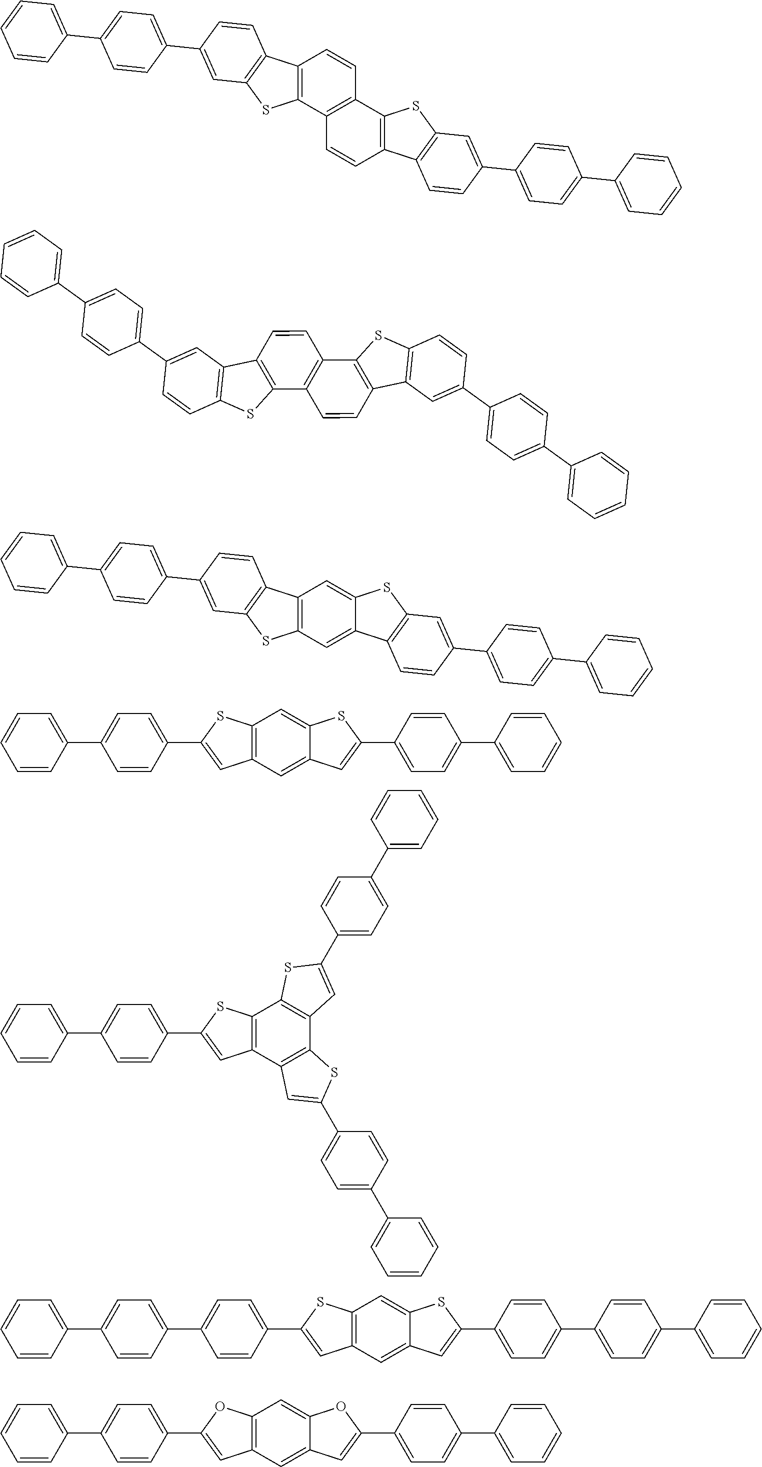

The difference in the ionization potential between the specific compound and the p-type organic semiconductor is preferably 0.1 eV or more.

In a case where the photoelectric conversion film contains the p-type organic semiconductor, a content of the p-type organic semiconductor in the photoelectric conversion film (=film thickness of p-type organic semiconductor in terms of single layer/film thickness of photoelectric conversion film×100) is preferably 15% to 75% by volume, more preferably 20% to 60% by volume, and still more preferably 25% to 50% by volume.

The p-type organic semiconductor may be used alone, or two or more thereof may be used in combination.

### <Coloring Agent>

The photoelectric conversion film may further include a coloring agent in addition to the specific compound.

The coloring agent is a compound different from the specific compound.

The coloring agent is preferably an organic coloring agent.

Examples of the coloring agent include a cyanine coloring agent, a styryl coloring agent, a hemicyanine coloring agent, a merocyanine coloring agent (including zeromethine merocyanine (simple merocyanine)), a rhodacyanine coloring agent, an allopolar coloring agent, an oxonol coloring agent, a hemioxonol coloring agent, a squarylium coloring agent, a croconium coloring agent, an azamethine coloring agent, a coumarin coloring agent, an arylidene coloring agent, an anthraquinone coloring agent, a triphenylmethane coloring agent, an azo coloring agent, an azomethine coloring agent, a metallocene coloring agent, a fluorenone coloring agent, a flugide coloring agent, a perylene coloring agent, a phenazine coloring agent, a phenothiazine coloring agent, a quinone coloring agent, a diphenylmethane coloring agent, a polyene coloring agent, an acridine coloring agent, a quinoxaline coloring agent, an acridinone coloring agent, a diphenylamine coloring agent, a quinophthalone coloring agent, a phenoxazine coloring agent, a phthaloperylene coloring agent, a dioxane coloring agent, a porphyrin coloring agent, a chlorophyll coloring agent, a phthalocyanine coloring agent, a subphthalocyanine coloring agent, a metal complex coloring agent, compounds disclosed in paragraphs [0083] to [0089] of JP2014-082483A, compounds disclosed in paragraphs [0029] to [0033] of JP2009-167348A, compounds disclosed in paragraphs [0197] to [0227] of JP2012-077064A, compounds disclosed in paragraphs [0035] to [0038] of WO2018-105269A, compounds disclosed in paragraphs [0041] to [0043] of WO2018-186389A, compounds disclosed in paragraphs [0059] to [0062] of WO2018-186397A, compounds disclosed in paragraphs [0078] to [0083] of WO2019-009249A, compounds disclosed in paragraphs [0054] to [0056] of WO2019-049946A, compounds disclosed in paragraphs [0059] to [0063] of WO2019-054327A, compounds disclosed in paragraphs [0086] to [0087] of WO2019-098161A, and compounds disclosed in paragraphs [0085] to [0114] of WO2020-013246.

A content of the coloring agent in the photoelectric conversion film (=film thickness of coloring agent in terms of single layer/film thickness of photoelectric conversion film×100) is preferably 1% to 85% by volume, more preferably 5% to 60% by volume, and still more preferably 10% to 40% by volume.

A content of the coloring agent with respect to the total content of the specific compound and the coloring agent in the photoelectric conversion film (=(film thickness of coloring agent in terms of single layer/(film thickness of specific compound in terms of single layer+film thickness of coloring agent in terms of single layer)×100)) is preferably 1% to 75% by volume, more preferably 5% to 65% by volume, and still more preferably 10% to 60% by volume.

The coloring agent may be used alone, or two or more kinds thereof may be used in combination.

The maximum absorption wavelength of the coloring agent is preferably within a range of 400 to 700 nm, and more preferably within a range of 450 to 650 nm.

The maximum absorption wavelength is a value measured in a solution state (solvent: chloroform) by an absorption spectrum of the coloring agent being adjusted to a concentration having an absorbance of about 0.5 to 1. However, in a case where the coloring agent is not dissolved in chloroform, a value measured by using the coloring agent in which the coloring agent is vapor-deposited and formed into a film state is defined as a maximum absorption wavelength of the coloring agent.

The photoelectric conversion film containing the specific compound is a non-light emitting film, and has a feature different from organic light emitting diodes (OLEDs). The non-light emitting film is intended for a film having a light emission quantum efficiency of 1% or less, and the light emission quantum efficiency is preferably 0.5% or less, and more preferably 0.1% or less. The lower limit thereof is often 0% or more.

### <Film Formation Method>

Examples of a film formation method of the photoelectric conversion film include a dry film formation method.

Examples of the dry film formation method include a physical vapor deposition method such as a vapor deposition method (particularly, a vacuum vapor deposition method), a sputtering method, an ion plating method, and a molecular beam epitaxy (MBE) method, and a chemical vapor deposition (CVD) method such as plasma polymerization, and the vacuum vapor deposition method is preferable. In a case where the photoelectric conversion film is formed by the vacuum vapor deposition method, manufacturing conditions such as a degree of vacuum and a vapor deposition temperature can be set according to the normal method.

The film thickness of the photoelectric conversion film is preferably 10 to 1000 nm, more preferably 50 to 800 nm, still more preferably 50 to 500 nm, and particularly preferably 50 to 300 nm.

### [Electrode]

The photoelectric conversion element preferably has an electrode.

Electrodes (the upper electrode (the transparent conductive film) 15 and the lower electrode (the conductive film) 11) are formed of conductive materials. Examples of the conductive material include metals, alloys, metal oxides, electrically conductive compounds, and mixtures thereof.

Since light is incident through the upper electrode 15, the upper electrode 15 is preferably transparent to light to be detected. Examples of the materials constituting the upper electrode 15 include conductive metal oxides such as tin oxide (antimony tin oxide (ATO) and fluorine doped tin oxide (FTO)) doped with antimony, fluorine or the like, tin oxide, zinc oxide, indium oxide, indium tin oxide (ITO), and indium zinc oxide (IZO); metal thin films such as gold, silver, chromium, and nickel; mixtures or laminates of these metals and the conductive metal oxides; and organic conductive materials such as polyaniline, polythiophene, and polypyrrole, and conductive metal oxides are preferable in terms of high conductivity and transparency.

Generally, in a case where the conductive film is made thinner than a certain range, the resistance value rapidly increases in many cases. In the solid-state imaging element in which the photoelectric conversion element according to the present embodiment is incorporated, the sheet resistance may be 100 to 10,000Ω/□, and the degree of freedom of the film thickness range that can be reduced is large.

In addition, as the film thickness of the upper electrode (the transparent conductive film) 15 is thinner, the amount of light that the upper electrode absorbs is smaller, and the light transmittance usually increases. The increase in the light transmittance causes an increase in light absorbance in the photoelectric conversion film and an increase in the photoelectric conversion ability, which is preferable. Considering the suppression of leakage current, an increase in the resistance value of the thin film, and an increase in transmittance accompanied by the thinning, the thickness of the upper electrode 15 is preferably 5 to 100 nm, and more preferably 5 to 20 nm.

There is a case where the lower electrode 11 has transparency or an opposite case where the lower electrode 11 does not have transparency and reflects light, depending on the application. Examples of a material constituting the lower electrode 11 include conductive metal oxides such as tin oxide (ATO, FTO) doped with antimony, fluorine, or the like, tin oxide, zinc oxide, indium oxide, indium tin oxide (ITO), and indium zinc oxide (IZO); metals such as gold, silver, chromium, nickel, titanium, tungsten, and aluminum, conductive compounds (for example, titanium nitride (TiN) and the like) such as oxides or nitrides of these metals; mixtures or laminates of these metals and conductive metal oxides; and organic conductive materials such as polyaniline, polythiophene, and polypyrrole.

The method of forming electrodes can be appropriately selected in accordance with the electrode material. Specific examples thereof include a wet method such as a printing method and a coating method; a physical method such as a vacuum vapor deposition method, a sputtering method, and an ion plating method; and a chemical method such as a CVD method and a plasma CVD method.

In a case where the material of the electrode is ITO, examples thereof include an electron beam method, a sputtering method, a resistance heating vapor deposition method, a chemical reaction method (such as a sol-gel method), and a coating method with a dispersion of indium tin oxide.

### [Charge Blocking Film: Electron Blocking Film and Positive Hole Blocking Film]

It is preferable that the photoelectric conversion element has one or more interlayers between the conductive film and the transparent conductive film, in addition to the photoelectric conversion film.

An example of the interlayer includes a charge blocking film. In a case where the photoelectric conversion element has this film, the characteristics (such as photoelectric conversion efficiency and responsiveness) of the photoelectric conversion element to be obtained are more excellent. Examples of the charge blocking film include an electron blocking film and a positive hole blocking film.

### <Electron Blocking Film>

The electron blocking film is a donor organic semiconductor material (a compound), and the p-type organic semiconductor described above can be used.

A polymer material can also be used as the electron blocking film.

Examples of the polymer material include a polymer such as phenylenevinylene, fluorene, carbazole, indole, pyrene, pyrrole, picoline, thiophene, acetylene, and diacetylene, and a derivative thereof.

The electron blocking film may be formed of a plurality of films.

The electron blocking film may be formed of an inorganic material. In general, since an inorganic material has a dielectric constant larger than that of an organic material, in a case where the inorganic material is used in the electron blocking film, a large voltage is applied to the photoelectric conversion film. Therefore, the photoelectric conversion efficiency increases. Examples of the inorganic material that can be used for the electron blocking film include calcium oxide, chromium oxide, copper chromium oxide, manganese oxide, cobalt oxide, nickel oxide, copper oxide, copper gallium oxide, copper strontium oxide, niobium oxide, molybdenum oxide, copper indium oxide, silver indium oxide, and iridium oxide.

### <Positive Hole Blocking Film>

A positive hole blocking film is an acceptor-property organic semiconductor material (a compound), and the n-type organic semiconductor described above can be used.

In addition, the positive hole blocking film may be formed with a plurality of films.

Examples of a method of producing a charge blocking film include a dry film formation method and a wet film formation method. Examples of the dry film formation method include a vapor deposition method and a sputtering method. The vapor deposition method may be any of a physical vapor deposition (PVD) method and a chemical vapor deposition (CVD) method, and the physical vapor deposition method such as a vacuum vapor deposition method is preferable. Examples of the wet film formation method include an ink jet method, a spray method, a nozzle printing method, a spin coating method, a dip coating method, a casting method, a die coating method, a roll coating method, a bar coating method, and a gravure coating method, and an ink jet method is preferable from the viewpoint of high accuracy patterning.

Each film thickness of the charge blocking films (the electron blocking film and the positive hole blocking film) is preferably 3 to 200 nm, more preferably 5 to 100 nm, and still more preferably 5 to 30 nm.

### [Substrate]

The photoelectric conversion element may further include a substrate.

Examples of the substrate include a semiconductor substrate, a glass substrate, and a plastic substrate.

The position of the substrate is not particularly limited, but in general, the conductive film, the photoelectric conversion film, and the transparent conductive film are laminated on the substrate in this order.

### [Sealing Layer]

The photoelectric conversion element may further include a sealing layer.

The performance of the photoelectric conversion material may deteriorate noticeably due to the presence of deterioration factors such as water molecules. The deterioration can be prevented by coating and sealing the entirety of the photoelectric conversion film with the sealing layer such as diamond-like carbon (DLC) or ceramics such as metal oxide, metal nitride, or metal nitride oxide which are dense and into which water molecules do not permeate.

Examples of the sealing layer include compounds described in paragraphs [0210] to [0215] of JP2011-082508A, the contents of which are incorporated herein by reference.

### [Imaging Element]

An example of the application of the photoelectric conversion element includes an imaging element.

The imaging element is an element that converts optical information of an image into an electric signal. In general, a plurality of the photoelectric conversion elements are arranged in a matrix on the same plane, and an optical signal is converted into an electric signal in each photoelectric conversion element (pixel) to sequentially output the electric signal to the outside of the imaging element for each pixel. Therefore, each pixel is formed of one or more photoelectric conversion elements and one or more transistors.

### [Optical Sensor]

Examples of another application of the photoelectric conversion element include the photoelectric cell and the optical sensor, but the photoelectric conversion element of the embodiment of the invention is preferably used as the optical sensor. The photoelectric conversion element may be used alone as the optical sensor. Alternately, the photoelectric conversion element may be used as a line sensor in which the photoelectric conversion elements are linearly arranged or as a two-dimensional sensor in which the photoelectric conversion elements are arranged on a plane.

### [Compound]

The present invention also relates to a compound.

The compound according to the embodiment of the present invention has the same definition as the specific compound described above, and the suitable embodiments thereof are also the same.

### EXAMPLES

Hereinafter, the present invention will be described in more detail with reference to Examples. The materials, amounts of use, proportions, treatments, procedures, and the like described in the following Examples can be modified as appropriate as long as the gist of the invention is maintained. Accordingly, the scope of the present invention should not be construed as being limited to Examples shown below.

### [Compound (Evaluation Compound)]

### <Synthesis of Compound (D-1)>

Compound (D-1) serving as a specific compound was synthesized according to the following scheme.

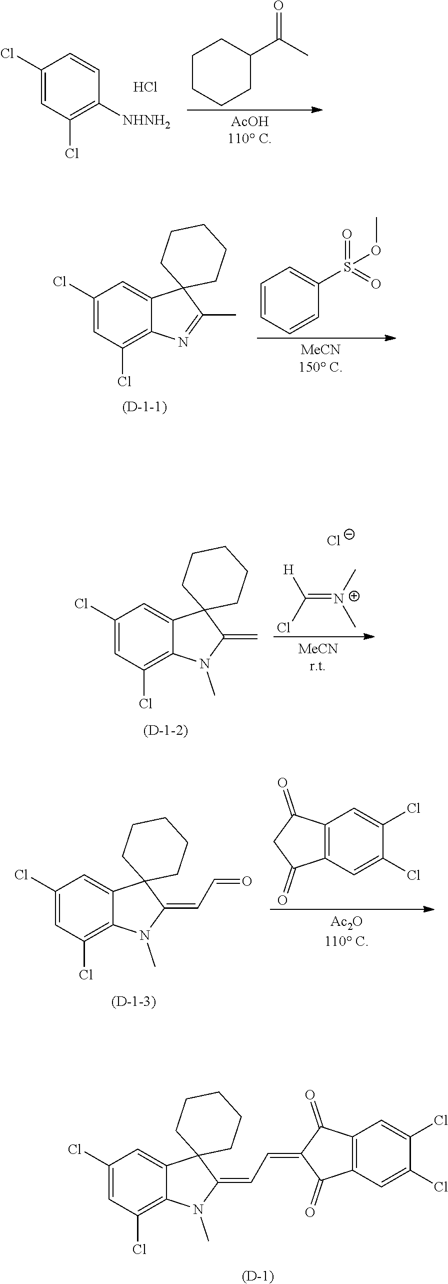

2,4-Dichlorophenyl hydrazine hydrochloride (10.0 g, 46.8 mmol), cyclohexyl methyl ketone (11.8 g, 93.7 mmol), and acetate (200 mL) were mixed and stirred at 110° C. for 4 hours. The mixture was allowed to cool to room temperature, and the solvent was distilled off under reduced pressure. The obtained residue was dissolved in ethyl acetate, and washed with water and saturated saline, and the obtained organic layer was dried over magnesium sulfate, then filtered, and concentrated under reduced pressure. The obtained crude product was purified by silica gel chromatography (eluent (volume ratio): hexane/ethyl acetate=1/1) to obtain Compound (D-1-1) (8.6 g, 68%).

Compound (D-1-1) (6.0 g, 22.5 mmol), methyl p-toluenesulfonate (8.7 g, 46.7 mmol), and acetonitrile (45 mL) were mixed and stirred at 150° C. for 2 hours using a microwave device. The reaction mixture was allowed to cool to room temperature, diluted with water (90 mL), then adjusted to pH 8 by the addition of 2 M sodium hydroxide aqueous solution, and extracted with ethyl acetate. The obtained organic layer was dried over magnesium sulfate, filtered, and concentrated under reduced pressure. The obtained crude product was purified by silica gel chromatography (eluent (volume ratio): hexane/ethyl acetate=9/1) to obtain Compound (D-1-2) (5.2 g, 83%).

(Chloromethylene) dimethyliminium chloride (7.10 g, 55.5 mmol) and acetonitrile (100 mL) were mixed, Compound (D-1-2) (5.2 g, 18.5 mmol) was added thereto, and the mixture was stirred at room temperature for 30 minutes. The reaction solution was added dropwise to a mixture of a 1 mol/L sodium hydroxide aqueous solution (150 mL) and ice (150 g), and the obtained mixed solution was stirred for 1 hour and extracted with ethyl acetate. The obtained organic layer was dried over magnesium sulfate, filtered, and concentrated under reduced pressure. The obtained crude product was purified by silica gel chromatography (eluent (volume ratio): dichloromethane/ethyl acetate=8/2) to obtain Compound (D-1-3) (4.2 g, 73%).

Compound (D-1-3) (1.0 g, 3.2 mmol), 5,6-dichloroindandione (0.95 g, 4.2 mmol), and acetic acid anhydride (20 mL) were mixed and stirred at 110° C. for 4 hours. The reaction solution was allowed to cool to room temperature, and then the resulting precipitate was filtered and washed with methanol. The obtained crude product was purified by silica gel chromatography (eluent (volume ratio): dichloromethane/ethyl acetate=95/5) to obtain Compound (D-1) (1.3 g, yield 77%).

Other specific compounds were also synthesized with reference to the above-described synthesis method.

The specific compound and comparative compound used in a test are shown below. Hereinbelow, Compounds (D-1) to (D-10) are specific compounds, and Compounds (R-1) and (R-2) are comparative compounds.

Hereinafter, the specific compound and the comparative compound are collectively referred to as Evaluation Compound.

Evaluation compounds were used for producing photoelectric conversion elements described later.

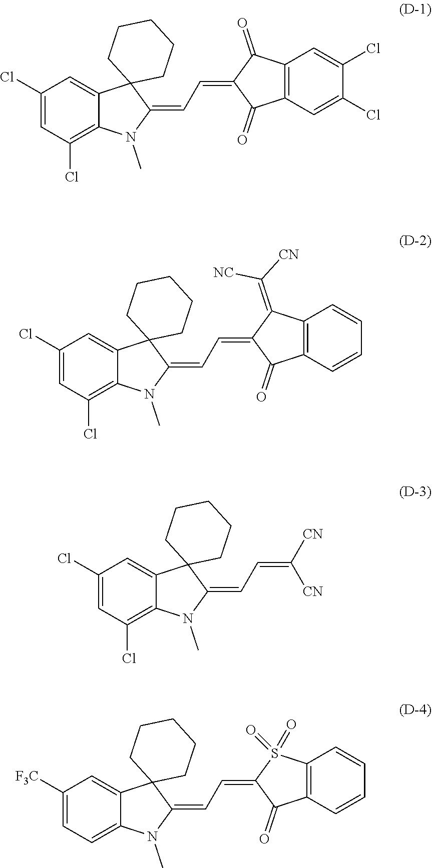 [SMILES: CN1/C(=C/C=C2C(=O)c3cc(Cl)c(Cl)cc3C2=O)C2(CCCCC2)c2cc(Cl)cc(Cl)c21 ; CN1/C(=C/C=C2\C(=O)c3ccccc3C2=C(C#N)C#N)C2(CCCCC2)c2cc(Cl)cc(Cl)c21 ; CN1/C(=C/C=C(C#N)C#N)C2(CCCCC2)c2cc(Cl)cc(Cl)c21 ; CN1/C(=C/C=C2\C(=O)c3ccccc3S2(=O)=O)C2(CCCCC2)c2cc(C(F)(F)F)ccc21]

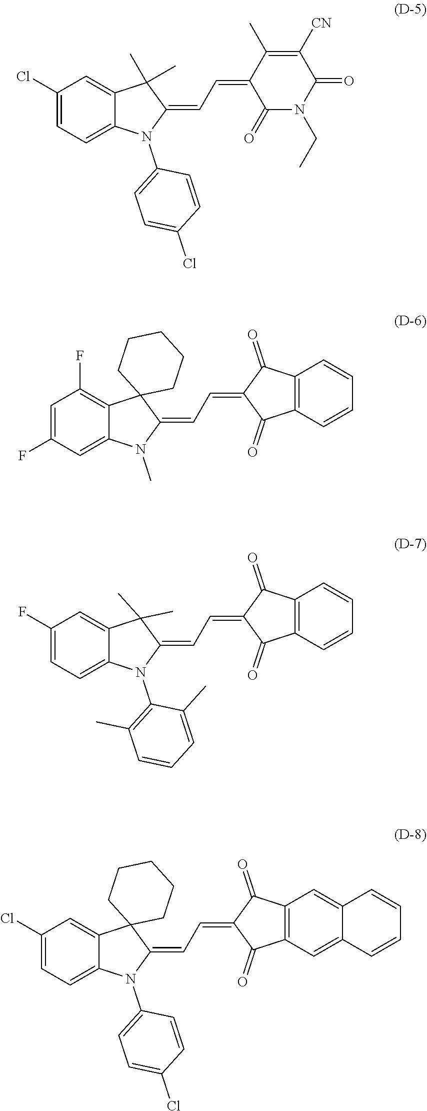 [SMILES: CCN1C(=O)C(C#N)=C(C)/C(=C/C=C2/N(c3ccc(Cl)cc3)c3ccc(Cl)cc3C2(C)C)C1=O ; CN1/C(=C/C=C2C(=O)c3ccccc3C2=O)C2(CCCCC2)c2c(F)cc(F)cc21 ; Cc1cccc(C)c1N1/C(=C/C=C2C(=O)c3ccccc3C2=O)C(C)(C)c2cc(F)ccc21 ; O=C1C(=C/C=C2/N(c3ccc(Cl)cc3)c3ccc(Cl)cc3C23CCCCC3)C(=O)c2cc3ccccc3cc21]

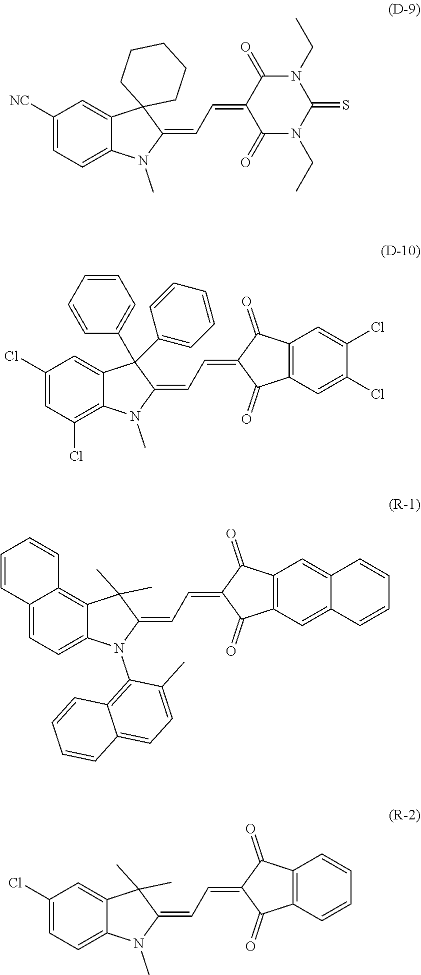 [SMILES: CCN1C(=O)C(=C/C=C2/N(C)c3ccc(C#N)cc3C23CCCCC3)C(=O)N(CC)C1=S ; CN1/C(=C/C=C2C(=O)c3cc(Cl)c(Cl)cc3C2=O)C(c2ccccc2)(c2ccccc2)c2cc(Cl)cc(Cl)c21 ; Cc1ccc2ccccc2c1N1/C(=C/C=C2C(=O)c3cc4ccccc4cc3C2=O)C(C)(C)c2c1ccc1ccccc21 ; CN1/C(=C/C=C2C(=O)c3ccccc3C2=O)C(C)(C)c2cc(Cl)ccc21]

[n-Type Organic Semiconductor Material]

Fullerene C₆₀ (C60) was used for the production of photoelectric conversion elements described later, as the n-type organic semiconductor used for evaluations.

[p-Type Organic Semiconductor]

The p-type organic semiconductor described below was used for producing the photoelectric conversion elements described later, as the p-type organic semiconductor used for evaluations.

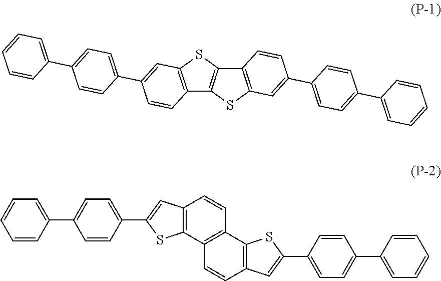 [SMILES: c1ccc(-c2ccc(-c3ccc4c(c3)sc3c5ccc(-c6ccc(-c7ccccc7)cc6)cc5sc43)cc2)cc1 ; c1ccc(-c2ccc(-c3cc4ccc5c(ccc6cc(-c7ccc(-c8ccccc8)cc7)sc65)c4s3)cc2)cc1]

### [Evaluation]

### <Production of Photoelectric Conversion Element>

A photoelectric conversion element having the form illustrated in FIG. 1 was produced using evaluation compounds (specific compounds or comparative compounds). Here, the photoelectric conversion element consists of a lower electrode 11, an electron blocking film 16A, a photoelectric conversion film 12, and an upper electrode 15.

Specifically, an amorphous ITO was formed into a film on a glass substrate by a sputtering method to form the lower electrode 11 (thickness: 30 nm). Furthermore, a compound (EB-1) described below was formed into a film on the lower electrode 11 by a vacuum thermal vapor deposition method to form the electron blocking film 16A (thickness: 30 nm).

Furthermore, in a state where the temperature of the substrate was controlled to 25° C., the evaluation compound, the n-type organic semiconductor material (fullerene (C₆₀)), and as desired, the p-type organic semiconductor material was co-vapor deposited on the electron blocking film 16A by a vacuum deposition method, each to be 80 nm in terms of a single layer, thereby forming a film. As a result, a photoelectric conversion film 12 having a bulk hetero structure of 160 nm (240 nm in a case where the p-type organic semiconductor material was also used) was formed. In this case, a film formation rate of the photoelectric conversion film 12 was set to 1.0 Å/sec.

Furthermore, amorphous ITO was formed into a film on the photoelectric conversion film 12 by a sputtering method to form the upper electrode 15 (the transparent conductive film) (the thickness: 10 nm). After the SiO film was formed as the sealing layer on the upper electrode 15 by a vacuum vapor deposition method, an aluminum oxide (Al₂O₃) layer was formed thereon by an atomic layer chemical vapor deposition (ALCVD) method to produce a photoelectric conversion element.

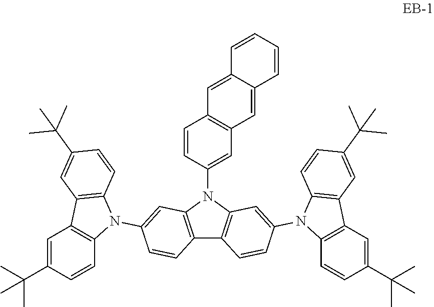 [SMILES: CC(C)(C)c1ccc2c(c1)c1cc(C(C)(C)C)ccc1n2-c1ccc2c3ccc(-n4c5ccc(C(C)(C)C)cc5c5cc(C(C)(C)C)ccc54)cc3n(-c3ccc4cc5ccccc5cc4c3)c2c1]

### <Relative Ratio of Dark Current>

10 elements were produced for each of Examples and Comparative Examples by the method described above, and the dark current of each of the obtained photoelectric conversion elements was measured by the following method. A voltage was applied to the lower electrode and the upper electrode of each of the photoelectric conversion elements to have an electric field strength of 2.5×10⁵ V/cm and current values (dark current) in a dark place were measured. The relative ratio of dark current was calculated from the following formula, and evaluation was carried out based on the following evaluation standard.

$$Relative ratio of dark current = ( value of highest dark current among 10 elements ) / ( value of lowest dark current among 10 elements )$$

### (Evaluation Standard)

“A”: relative ratio of dark current is less than 2.0
“B”: relative ratio of dark current is 2.0 or more and less than 3.0
“C”: relative ratio of dark current is 3.0 or more and less than 4.0
“D”: relative ratio of dark current is 4.0 or more

### <Evaluation of Photoelectric Conversion Efficiency (External Quantum Efficiency)>

The drive of each photoelectric conversion element thus obtained was confirmed. A voltage was applied to each photoelectric conversion element to have an electric field strength of 2.0×10⁵ V/cm. Thereafter, light is emitted from the upper electrode (transparent conductive film) side to perform an incident photon-to-current conversion efficiency (IPCE) measurement, and the external quantum efficiency (external quantum efficiency before the continuous drive) at 540 nm was extracted.

It was confirmed that all of the photoelectric conversion elements produced by using the compounds in Examples and compounds in Comparative Examples (Compounds (D-1) to (D-10), (R-1), and (R-2) exhibited a photoelectric conversion efficiency of 50% or more and had the sufficient external quantum efficiency as the photoelectric conversion elements. The external quantum efficiency was measured using a constant energy quantum efficiency measuring device manufactured by Optel Co., Ltd. In addition, the amount of light emitted was 50 μW/cm².

### <Evaluation of Heat Resistance>

The heat resistance of each of the obtained photoelectric conversion elements was evaluated according to the following method.

Specifically, each of the obtained photoelectric conversion elements was heated on a hot plate at 180° C. for 30 minutes. A voltage was applied to each photoelectric conversion element after heating so that the electric field strength was 2.0×10⁵ V/cm, IPCE measurement was performed by irradiating light from the upper electrode (the transparent conductive film) side, and external quantum efficiency at 540 nm (external quantum efficiency before continuous drive) was extracted. The external quantum efficiency was measured using a constant energy quantum efficiency measuring device manufactured by Optel Co., Ltd. In addition, the amount of light emitted was 50 ρW/cm².

Next, the maintenance amount of the external quantum efficiency was calculated from the following expression. The obtained numerical value was evaluated as an index of heat resistance according to the following evaluation standards.

In the following expression, the “external quantum efficiency (%) before heating” means a value measured in a case where the above-described external quantum efficiency measurement is performed on each photoelectric conversion element without performing the above-described heating treatment.

Maintenance amount of external quantum efficiency=(external quantum efficiency (%) after heating/external quantum efficiency (%) before heating)
“A”: maintenance amount of external quantum efficiency is 0.95 or more
“B”: maintenance amount of external quantum efficiency is 0.9 or more and less than 0.95
“C”: maintenance amount of external quantum efficiency is 0.8 or more and less than 0.9
“D”: maintenance amount of external quantum efficiency was less than 0.8

Hereinafter, Table 1 is shown below.

The “HOMO (eV) of B¹” in the table represents HOMO energy levels obtained by performing structure-optimization calculation by density-functional calculation B3LYP/6-31G(d) using quantum chemical calculation software Gaussian09 with respect to the compound represented by Formula (B-1) described above, which is a model compound of each of Evaluation Compounds (D-1) to (D-10) and (R-1) and (R-2).

TABLE 1

| | Feature component of | | | | | |
|---|---|---|---|---|---|---|
| | photoelectric conversion film | | | | Evaluation | |
| | | n-Type organic | p-Type organic | HOMO | Relative | |
| | Evaluation | semiconductor | semiconductor | (eV) | ratio of | Heat |
| | Compound | material | material | of B¹ | dark current | resistance |
| Example 1 | (D-1) | C60 | — | −5.22 | A | B |
| Example 2 | (D-2) | C60 | — | −5.22 | A | B |
| Example 3 | (D-3) | C60 | — | −5.22 | A | B |
| Example 4 | (D-4) | C60 | — | −5.21 | A | B |
| Example 5 | (D-5) | C60 | — | −4.99 | B | B |
| Example 6 | (D-6) | C60 | — | −5.15 | A | B |
| Example 7 | (D-7) | C60 | — | −4.85 | C | B |
| Example 8 | (D-8) | C60 | — | −4.99 | B | A |
| Example 9 | (D-9) | C60 | — | −5.37 | A | B |
| Example 10 | (D-10) | C60 | — | −5.22 | A | C |
| Example 11 | (D-1) | C60 | (P-1) | −5.22 | A | A |
| Example 12 | (D-1) | C60 | (P-2) | −5.22 | A | A |
| Comparative | (R-1) | C60 | — | −4.69 | D | B |
| Example 1 | | | | | | |
| Comparative | (R-2) | C60 | — | −4.99 | B | D |
| Example 2 | | | | | | |
| Comparative | (R-1) | C60 | (P-1) | −4.69 | D | A |
| Example 3 | | | | | | |

From the results in Table 1, it is clear that the photoelectric conversion elements of Examples have suppressed variations in dark current and have excellent heat resistance.

In addition, from the comparison of Examples, it was confirmed that in a case where B¹ in the specific compound has a structure in which the HOMO energy calculated using a compound represented by Formula (B-1) described later as a model compound is −4.90 eV or less (preferably −5.000 eV or less and more preferably −5.10 eV or less), the variation in dark current is further suppressed.

In addition, from the comparison of Examples, it was confirmed that in a case where R³ in the specific compound represents a linear or branched alkyl group which has a molecular weight of 160 or less and may have a substituent, and R⁴ and R⁵ are bonded to each other to form a ring not including an oxygen atom as a ring member atom, the heat resistance is more excellent (particularly, see the results of Example 10).

In addition, from the comparison of Examples, it was confirmed that in a case where A¹ in the specific compound represents the group represented by Formula (A-3), and R^{Y9} and R^{Y10} are bonded to each other to form a ring, the heat resistance is more excellent (particularly, see the results of Example 8).

### EXPLANATION OF REFERENCES

10a, 10b: photoelectric conversion element
11: conductive film (lower electrode)
12: photoelectric conversion film
15: transparent conductive film (upper electrode)
16A: electron blocking film
16B: positive hole blocking film

What is claimed is:

1. A photoelectric conversion element comprising, in the following order:
a conductive film;
a photoelectric conversion film; and
a transparent conductive film,
wherein the photoelectric conversion film contains a compound represented by Formula (1), 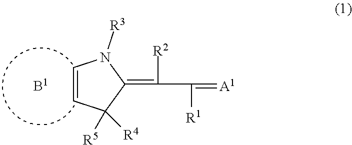
in Formula (1), R¹ and R² each independently represent a hydrogen atom or a substituent,
R³ represents a linear or branched alkyl group which has a molecular weight of 160 or less and may have a substituent, a cycloalkyl group which may have a substituent, an aryl group which may have a substituent, or a heteroaryl group which may have a substituent,
R⁴ and R⁵ each independently represent a substituent, and R⁴ and R⁵ may be bonded to each other to form a ring, provided that in a case where R³ represents a linear or branched alkyl group which has a molecular weight of 160 or less and may have a substituent, R⁴ and R⁵ each independently represent a cycloalkyl group which may have a substituent, an aryl group which may have a substituent, or a heteroaryl group which may have a substituent, or R⁴ and R⁵ are bonded to each other to form a ring containing no oxygen atom as a ring member atom,
A¹ represents a group represented by Formula (A-1) or a group represented by Formula (A-2), 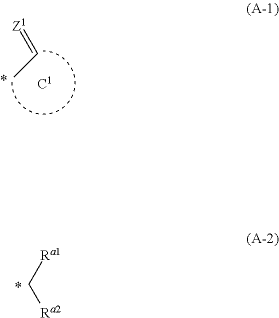
in Formula (A-1), * represents a bonding position,
C¹ represents a ring which contains at least two carbon atoms and may have a substituent,
Z¹ represents an oxygen atom, a sulfur atom, —NR^{Z1}, or —CR^{Z2}R^{Z3}, where R^{Z1} represents a hydrogen atom or a substituent, R^{Z2} and R^{Z3} each independently represent a cyano group, —SO₂R^{Z4}, —COOR^{Z5}, or —COR^{Z6}, and R^{Z4}, R^{Z5}, and R^{Z6} each independently represent an alkyl group which may have a substituent, an aryl group which may have a substituent, or a heteroaryl group which may have a substituent,
in Formula (A-2), * represents a bonding position,
R^{a1} and R^{a2} each independently represent a cyano group, —SO₂R^{b1}, —COOR^{b2}, or —COR^{b3}, where R^{b1}, R^{b2}, and R^{b3} each independently represent an alkyl group which may have a substituent, an aryl group which may have a substituent, or a heteroaryl group which may have a substituent, and
B¹ represents a benzene ring having a substituent, provided that B¹ satisfies Condition BX,
<Condition BX>
a compound represented by Formula (B-1) has HOMO energy of less than −4.80 eV, which is obtained by performing structure-optimization calculation according to density-functional calculation B3LYP/6-31G(d) using quantum chemical calculation software Gaussian09, 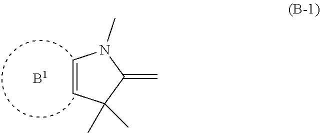
B¹ in Formula (B-1) represents a benzene ring having a substituent, and B¹ in Formula (B-1) is the same as B¹ in Formula (1).

2. The photoelectric conversion element according to claim 1,
wherein the substituent in B¹ includes no substituent having a Hammett's substituent constant 6p of 0.05 or less.

3. The photoelectric conversion element according to claim 1,
wherein A¹ represents the group represented by Formula (A-1).

4. The photoelectric conversion element according to claim 1,
wherein A¹ represents a group represented by Formula (A-3), 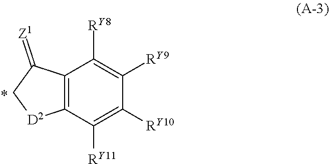
in Formula (A-3), * represents a bonding position,
Z¹ represents an oxygen atom, a sulfur atom, ═NR^{Z1}, or ═CR^{Z2}R^{Z3}, where R^{Z1} represents a hydrogen atom or a substituent, R^{Z2} and R^{Z3} each independently represent a cyano group, —SO₂R^{Z4}, —COOR^{Z5}, or —COR^{Z6}, and R^{Z4}, R^{Z5}, and R^{Z6} each independently represent an alkyl group which may have a substituent, an aryl group which may have a substituent, or a heteroaryl group which may have a substituent,
D² represents —O—, —S—, —SO₂—, —CO—, —CS—, —C(═NR^{D1})—, or —C(═CR^{D2}R^{D3})—, where R^{D1} represents a hydrogen atom or a substituent, R^{D2} and R^{D3} each independently represent a cyano group, —SO₂R^{D6}, —COOR^{D7}, or —COR^{D8}, and R^{D6}, R^{D7}, and R^{D8} each independently represent an alkyl group which may have a substituent, an aryl group which may have a substituent, or a heteroaryl group which may have a substituent, and
R^{Y8} to R^{Y11} each independently represent a hydrogen atom or a substituent, where R^{Y8} and R^{Y9}, R^{Y9} and R^{Y10}, and R^{Y10} and R^{Y11} may be bonded to each other to form a ring.

5. The photoelectric conversion element according to claim 1,
wherein Z¹ represents an oxygen atom.

6. The photoelectric conversion element according to claim 1,
wherein the photoelectric conversion film further contains an n-type organic semiconductor, and
the photoelectric conversion film has a bulk hetero structure formed in a state where the compound represented by Formula (1) and the n-type organic semiconductor are mixed.

7. The photoelectric conversion element according to claim 6,
wherein the n-type organic semiconductor includes fullerenes selected from the group consisting of a fullerene and a derivative of the fullerene.

8. The photoelectric conversion element according to claim 1,
wherein the photoelectric conversion film further contains a p-type organic semiconductor.

9. The photoelectric conversion element according to claim 1,
wherein the photoelectric conversion film further contains a coloring agent.

10. The photoelectric conversion element according to claim 1, further comprising:
one or more interlayers between the conductive film and the transparent conductive film, in addition to the photoelectric conversion film.

11. An imaging element comprising:
the photoelectric conversion element according to claim 1.

12. An optical sensor comprising:
the photoelectric conversion element according to claim 1.

13. A compound represented by Formula (1), 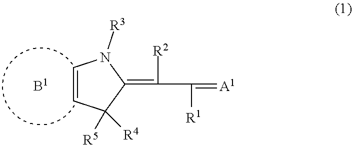
in Formula (1), R¹ and R² each independently represent a hydrogen atom or a substituent,
R³ represents a linear or branched alkyl group which has a molecular weight of 160 or less and may have a substituent, a cycloalkyl group which may have a substituent, an aryl group which may have a substituent, or a heteroaryl group which may have a substituent,
R⁴ and R⁵ each independently represent a substituent, where R⁴ and R⁵ may be bonded to each other to form a ring, provided that in a case where R³ represents a linear or branched alkyl group which has a molecular weight of 160 or less and may have a substituent, R⁴ and R⁵ each independently represent a cycloalkyl group which may have a substituent, an aryl group which may have a substituent, or a heteroaryl group which may have a substituent, or R⁴ and R⁵ are bonded to each other to form a ring containing no oxygen atom as a ring member atom,
A¹ represents a group represented by Formula (A-1) or a group represented by Formula (A-2), 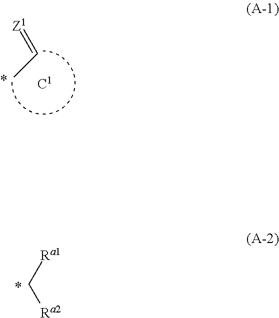
in Formula (A-1), * represents a bonding position,
C¹ represents a ring which contains at least two carbon atoms and may have a substituent,
Z¹ represents an oxygen atom, a sulfur atom, ═NR^{Z1}, or ═CR^{Z2}R^{Z3}, where R^{Z1} represents a hydrogen atom or a substituent, R^{Z2} and R^{Z3} each independently represent a cyano group, —SO₂R^{Z4}, —COOR^{Z5}, or —COR^{Z6}, and R^{Z4}, R^{Z5}, and R^{Z6} each independently represent an alkyl group which may have a substituent, an aryl group which may have a substituent, or a heteroaryl group which may have a substituent,
in Formula (A-2), * represents a bonding position,
R^{a1} and R^{a2} each independently represent a cyano group, —SO₂R^{b1}, —COOR^{b2}, or —COR^{b3}, where R^{b1}, R^{b2}, and R^{b3} each independently represent an alkyl group which may have a substituent, an aryl group which may have a substituent, or a heteroaryl group which may have a substituent, and
B¹ represents a benzene ring having a substituent, provided that B¹ satisfies Condition BX,
<Condition BX>
a compound represented by Formula (B-1) has HOMO energy of less than −4.80 eV, which is obtained by performing structure-optimization calculation according to density-functional calculation B3LYP/6-31G(d) using quantum chemical calculation software Gaussian09, 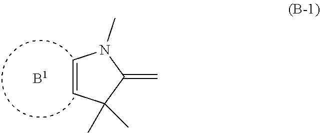
B¹ in Formula (B-1) represents a benzene ring having a substituent, and B¹ in Formula (B-1) is the same as B¹ in Formula (1).

14. The compound according to claim 13,
wherein the substituent in B¹ includes no substituent having a Hammett's substituent constant 6p of 0.05 or less.

15. The compound according to claim 13,
wherein A¹ represents the group represented by Formula (A-1).

16. The compound according to claim 13,
wherein A¹ represents a group represented by Formula (A-3), 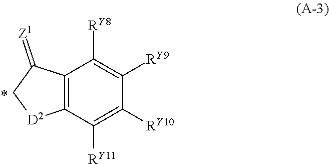
in Formula (A-3), * represents a bonding position,
Z¹ represents an oxygen atom, a sulfur atom, ═NR^{Z1}, or ═CR^{Z2}R^{Z3}, where R^{Z1} represents a hydrogen atom or a substituent, R^{Z2} and R^{Z3} each independently represent a cyano group, —SO₂R^{Z4}, —COOR^{Z5}, or —COR^{Z6}, and R^{Z4}, R^{Z5}, and R^{Z6} each independently represent an alkyl group which may have a substituent, an aryl group which may have a substituent, or a heteroaryl group which may have a substituent,
D² represents —O—, —S—, —SO₂—, —CO—, —CS—, —C(═NR^{D1})—, or —C(═CR^{D2}R^{D3})—, where R^{D1} represents a hydrogen atom or a substituent, R^{D2} and R^{D3} each independently represent a cyano group, —SO₂R^{D6}, —COOR^{D7}, or —COR^{D8}, and R^{D6}, R^{D7}, and R^{D8} each independently represent an alkyl group which may have a substituent, an aryl group which may have a substituent, or a heteroaryl group which may have a substituent, and
R^{Y8} to R^{Y11} each independently represent a hydrogen atom or a substituent, where R^{Y8} and R^{Y9}, R^{Y9} and R^{Y10}, and R^{Y10} and R^{Y11} may be bonded to each other to form a ring.

17. The compound according to claim 13,
wherein Z¹ represents an oxygen atom.

18. The photoelectric conversion element according to claim 2,
wherein A¹ represents the group represented by Formula (A-1).

19. The photoelectric conversion element according to claim 2,
wherein A¹ represents a group represented by Formula (A-3), 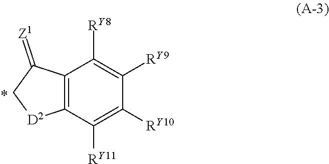
in Formula (A-3), * represents a bonding position,
Z¹ represents an oxygen atom, a sulfur atom, ═NR^{Z1}, or ═CR^{Z2}R^{Z3}, where R^{Z1} represents a hydrogen atom or a substituent, R^{Z2} and R^{Z3} each independently represent a cyano group, —SO₂R^{Z4}, —COOR^{Z5}, or —COR^{Z6}, and R^{Z4}, R^{Z5}, and R^{Z6} each independently represent an alkyl group which may have a substituent, an aryl group which may have a substituent, or a heteroaryl group which may have a substituent,
D² represents —O—, —S—, —SO₂—, —CO—, —CS—, —C(═NR^{D1})—, or —C(═CR^{D2}R^{D3})—, where R^{D1} represents a hydrogen atom or a substituent, R^{D2} and R^{D3} each independently represent a cyano group, —SO₂R^{D6}, —COOR^{D7}, or —COR^{D8}, and R^{D6}, R^{D7}, and R^{D8} each independently represent an alkyl group which may have a substituent, an aryl group which may have a substituent, or a heteroaryl group which may have a substituent, and
R^{Y8} to R^{Y11} each independently represent a hydrogen atom or a substituent, where R^{Y8} and R^{Y9}, R^{Y9} and R^{Y10}, and R^{Y10} and R^{Y11} may be bonded to each other to form a ring.

20. The photoelectric conversion element according to claim 2,
wherein Z¹ represents an oxygen atom.
